SEQUENCE LISTING (29 sequences)

SEQ ID NO: 1 - RNA, 23 nt, Homo sapiens
  Target sequence at exon 10 (rs12895357)(C)
agcagcagcagcgggaccuauca

SEQ ID NO: 2 - RNA, 22 nt, Homo sapiens
  miR357C.22
ugauaggucccgcugcugcugc

SEQ ID NO: 3 - RNA, 21 nt, Homo sapiens
  miR357C.21
ugauaggucccgcugcugcug

SEQ ID NO: 4 - RNA, 19 nt, Homo sapiens
  miR357C.19
ugauaggucccgcugcugc

SEQ ID NO: 5 - RNA, 20 nt, Homo sapiens
  miR357C.20
ugauaggucccgcugcugcu

SEQ ID NO: 6 - RNA, 23 nt, Homo sapiens
  miR357C.23
ugauaggucccgcugcugcugcu

SEQ ID NO: 7 - RNA, 23 nt, Homo sapiens
  Target sequence at exon 10 (rs12895357)(G)
agcagcagcagggggaccuauca

SEQ ID NO: 8 - RNA, 22 nt, Homo sapiens
  miR357G.22
ugauaggucccccugcugcugc

SEQ ID NO: 9 - RNA, 19 nt, Homo sapiens
  miR357G.19
ugauaggucccccugcugc

SEQ ID NO: 10 - RNA, 20 nt, Homo sapiens
  miR357G.20
ugauaggucccccugcugcu

SEQ ID NO: 11 - RNA, 21 nt, Homo sapiens
  miR357G.21
ugauaggucccccugcugcug

SEQ ID NO: 12 - RNA, 23 nt, Homo sapiens
  miR357G.23
ugauaggucccccugcugcugcu

SEQ ID NO: 13 - RNA, 23 nt, Homo sapiens
  Target sequence at exon 8 (rs1048755) (A)
accuggaacgaauguuagaagca

SEQ ID NO: 14 - RNA, 22 nt, Homo sapiens
  miR755A.22
ugcuucuaacauucguuccagg

SEQ ID NO: 15 - RNA, 19 nt, Homo sapiens
  miR755A.19
ugcuucuaacauucguucc

SEQ ID NO: 16 - RNA, 20 nt, Homo sapiens
  miR755A.20
ugcuucuaacauucguucca

SEQ ID NO: 17 - RNA, 21 nt, Homo sapiens
  miR755A.21
ugcuucuaacauucguuccag

SEQ ID NO: 18 - RNA, 23 nt, Homo sapiens
  miR755A.23
ugcuucuaacauucguuccaggu

SEQ ID NO: 19 - RNA, 23 nt, Homo sapiens
  Target sequence at exon 8 (rs1048755) (G)
accuggaacgaguguuagaagca

SEQ ID NO: 20 - RNA, 22 nt, Homo sapiens
  miR755G.22
ugcuucuaacacucguuccagg

SEQ ID NO: 21 - RNA, 19 nt, Homo sapiens
  miR755G.19
ugcuucuaacacucguucc

SEQ ID NO: 22 - RNA, 20 nt, Homo sapiens
  miR755G.20
ugcuucuaacacucguucca

SEQ ID NO: 23 - RNA, 21 nt, Homo sapiens
  miR755G.21
ugcuucuaacacucguuccag

SEQ ID NO: 24 - RNA, 23 nt, Homo sapiens
  miR755G.23
ugcuucuaacacucguuccaggu

SEQ ID NO: 25 - RNA, 21 nt, Homo sapiens
  miR-Control
caacaagaugaagagcaccaa

SEQ ID NO: 26 - RNA, 19 nt, Homo sapiens
  sh-ATXN3
gauaggucccgcugcugcu

SEQ ID NO: 27 - RNA, 28 nt, Homo sapiens
  miR155 - 5′ arm
cuggaggcuugcugaaggcuguaugcug

SEQ ID NO: 28 - RNA, 19 nt, Homo sapiens
  miR loop
guuuuggccacugacugac

SEQ ID NO: 29 - RNA, 43 nt, Homo sapiens
  miR155 - 3′ arm
caggacaaggccuguuacuagcacucacauggaacaaauggcc

(19) United States
(12) Patent Application Publication
(10) Pub. No.: US 2022/0098592 A1
(43) Pub. Date: Mar. 31, 2022

(54) DOUBLE STRANDED RNA AND USES THEREOF

(71) Applicants: UNIVERSIDADE DE COIMBRA, Coimbra (PT); CENTRO DE NEUROCIENCIAS E BIOLOGIA CELULAR, Coimbra (PT)

(72) Inventors: Rui Jorge GONÇALVES PEREIRA NOBRE, Coimbra (PT); Luís Fernando MORGADO PEREIRA DE ALMEIDA, Coimbra (PT)

(21) Appl. No.: 17/422,083

(22) PCT Filed: Jan. 9, 2020
(86) PCT No.: PCT/IB2020/050141

§ 371 Date: Jul. 9, 2021

(30) Foreign Application Priority Data
Jan. 9, 2019 (PT) 115253

(51) Int. Cl. C12N 15/113; C12N 15/86

(52) U.S. Cl. CPC C12N 15/1137; C12N 15/86; C12N 2310/141; C12N 2320/34; C12N 2750/14143

(57) ABSTRACT

The present disclosure relates to a non-invasive and allele-specific treatment, in particular for Machado-Joseph disease (MJD). The present disclosure uses RNA silencing technology (e.g. RNA interference) against exonic single nucleotide polymorphisms (SNPs) in the ataxin-3 gene, encoding the dominant gain-of-function mutant ataxin-3 protein, thereby resulting in an effective treatment for MJD. For that purpose, highly-target specific gene silencing RNAs, whose anti-sense sequences are complementary to SNPs that are in linkage disequilibrium with the disease-causing expansion, were designed and tested. Furthermore, this disclosure also relates to a selected adeno-associated viral vector, in particular serotype 9 (AAV9) as a gene delivery vector, upon which the said double stranded RNAs can be delivered into the central nervous system (CNS) by minimally invasive routes (e.g. intravenous administration), since this particular serotype efficiently crosses the blood-brain barrier (BBB).

### TECHNICAL FIELD

The present disclosure relates to a non-invasive and allele-specific treatment, in particular for Machado-Joseph disease (MJD). The present disclosure uses RNA silencing technology (e.g. RNA interference) against exonic single nucleotide polymorphisms (SNPs) in the ataxin-3 gene, encoding the dominant gain-of-function mutant ataxin-3 protein, thereby resulting in an effective treatment for MJD. For that purpose, highly-target specific gene silencing RNAs, whose anti-sense sequences are complementary to SNPs that are in linkage disequilibrium with the disease-causing expansion, were designed and tested.

Furthermore, this disclosure also relates to a selected adeno-associated viral vector, in particular serotype 9 (AAV9) as a gene delivery vector, upon which the said double stranded RNAs can be delivered into the central nervous system (CNS) by minimally invasive routes (e.g. intravenous administration), since this particular serotype efficiently crosses the blood-brain barrier (BBB).

### BACKGROUND

Machado-Joseph disease (MJD) is a dominant autosomal neurodegenerative disorder characterized by cerebellar dysfunction and loss of motor coordination. This disorder, which corresponds to the most common type of spinocerebellar ataxia worldwide, is caused by a genetic mutation in the coding region of the ataxin-3 gene (MJD1/ATXN3 gene). The genetic mutation involves a DNA segment of the ataxin-3 gene known as the CAG trinucleotide repeat. Normally, the CAG segment in the ataxin-3 gene of humans is repeated multiple times, i.e. about 10-42 times. People that develop MJD have an expansion of the number of CAG repeats in at least one allele. An affected person usually inherits the mutated allele from one affected parent. People with more than 51 CAG repeats may develop signs and symptoms of MJD, while people with 60 or more repeats almost always develop the disorder. The increase in the size of the CAG repeat leads to the production of an elongated (mutated) ataxin-3 protein. This protein is processed in the cell into smaller fragments that are cytotoxic and that accumulate and aggregate in neurons. This triggers multiple pathogenic mechanisms, ultimately leading to neurodegeneration in several brain regions, which underlies the signs and symptoms of MJD.

One of the most direct, specific and effective solutions to correct MJD would be to inhibit mutant ataxin-3 expression using RNA interference (RNAi), thus targeting the initial cause of the disorder. RNAi is a naturally occurring mechanism that involves sequence specific down-regulation of messenger RNA (mRNA). The down-regulation of mRNA results in a reduction of the amount of protein that is expressed. RNAi is triggered by double stranded RNA (dsRNAs). One of the strands of the dsRNA is substantially or completely complementary to its target, the mRNA. This strand is termed the guide strand, or anti-sense strand. The mechanism of RNAi involves the incorporation of the guide strand in the RNA-induced silencing complex (RISC). In this process, RISC prefers the strand whose 5′ end more loosely pairs with its complement. RISC is a multiple turnover complex that via complementary base pairing binds to its target mRNA. Once bound to its target mRNA it can either cleave the mRNA or reduce translation efficiency. RISC can cleave mRNA between residues paired to nucleotides 10 and 11 of the guide strand. RNAi has since its discovery been widely used to knock down specific target genes. The triggers for inducing RNAi that have been employed involve the use of small interfering RNA (siRNA) or short hairpin RNA (shRNA). In addition, molecules that can naturally trigger RNAi, the so-called micro RNAs (miRNAs), have been used to make artificial miRNAs that mimic their naturally occurring counterparts. These strategies have in common that they provide for dsRNA molecules that are designed to target a gene of choice. RNAi based therapeutic approaches that utilize the sequence specific modality of RNAi are under development and several are currently in clinical trials.

RNA interference has been employed to target both mutant and non-mutant ataxin-3 genes (WO2005105995, Alves et al., 2010). In the latter case, knockdown of the normal ataxin-3 protein in rats was shown not to have any apparent detrimental effects. Nevertheless, it is unknown whether neural cells in the human brain will tolerate long-term silencing of both mutant and non-mutant ataxin-3 genes. Therefore, efforts to either regulate silencing, or inhibit only the mutant allele should be explored, as decades-long therapy will be required for MJD.

One of the most specific and effective solutions would be to target SNPs located in the coding region of ataxin-3 gene, particularly SNP base nucleotides which are in linkage disequilibrium with the disease allele. For instance, the cytosine (C) in the SNP located at the 3′ end of the expanded CAG tract (C⁹⁸⁷GG/G⁹⁸⁷GG: r512895357) has been described as being in linkage disequilibrium with the disease, being associated with abnormal CAG expansion in 70% of MJD patients worldwide.

Allele-specific reduction of the mutant ataxin-3 gene has been investigated in cells (U.S. Ser. No. 10/072,264B2) and in rodent models of MJD (Alves et al., 2008a, Nobrega et al., 2013), by using siRNAs or shRNAs directed to cytosine (C) at r512895357. However, in these previous studies, designed sequences did not allow a complete allele-specific silencing of mutant allele. Moreover, the toxicity of silencing sequences in the central nervous system (CNS) of rodent models was not assessed in a durable treatment or in wild-type animals. In fact, it has been recently reported that shRNAs can lead to severe brain toxicity in long-term treatments or when high doses are used. Toxic side-effects have been associated with saturation of the cellular RNAi machinery and changes in endogenous miRNA expression. Moreover, previous allele-specific and viral-based silencing of mutant ataxin-3 in rodent models involved craniotomy and direct administration of viral vectors into the brain parenchyma, which is an invasive procedure, associated with potential adverse effects and results in limited vector dispersion throughout the brain, thereby not targeting all regions affected in MJD.

These facts are disclosed in order to illustrate the technical problem addressed by the present disclosure.

### GENERAL DESCRIPTION

As MJD involves the expression of a mutant ataxin-3 protein, the accumulation thereof leading to disease, RNAi provides for an opportunity to treat the disease as it can reduce expression of the ataxin-3 genes. The paradigm underlying this approach involves a reduction of the levels of mutant ataxin-3 mRNA, while preserving the normal ataxin-3 mRNA, to thereby reduce the toxic effects resulting from the mutant ataxin-3 protein, to achieve a reduction and/or delay of MJD symptoms, or even to prevent MJD symptoms altogether.

The present disclosure provides for SNP-targeting dsRNAs comprising a first RNA sequence and a second RNA sequence, wherein the first and second RNA sequence are substantially complementary, wherein the first RNA sequence has a sequence length of at least 19 nucleotides, preferably has a sequence length of 19-23 nucleotides and is complementary to SEQ ID NO. 1, 7, 13 or 19. Said dsRNAs are for use in inducing target-specific RNAi against human mutant ataxin-3 genes.

SNP-targeting dsRNAs of this disclosure involve targeting of SNPs that are present in two coding regions of disease alleles, i.e. r512895357 (exon 10) and r51048755 (exon 8) (FIG. 1). Such dsRNAs may be delivered alone or in combination, in a cell, either directly via transfection or indirectly via delivery of DNA (e.g. transfection) or via vector-mediated expression upon which the said dsRNAs can be expressed, to specifically target and reduce expression of mutated ataxin-3 genes that comprise a cytosine (C) (SEQ ID NO. 2, 3, 4, 5, and 6) or a guanine (G) (SEQ ID NO. 8, 9, 10, 11, and 12) at the r512895357 (C⁹⁸⁷GG/G⁹⁸⁷GG)—exon 10; or an adenine (A) (SEQ ID NO. 14, 15, 16, 17 and 18) or a guanine (G) (SEQ ID NO. 20, 21, 22, 23, and 24) at the r51048755 (A⁶⁶⁹TG/G⁶⁶⁹TG)—exon 8. Alternatively, SNP-targeting dsRNAs can also be used in combination to target both non-mutant and mutant ataxin-3 genes.

In particular, one of the designed SNP-targeting dsRNAs of the present disclosure, whose the first strand/sequence is SEQ ID NO. 2, was capable of reducing mutant ataxin-3 mRNA and protein levels when provided in a miRNA scaffold, by targeting the C nucleotide at the r512895357. This dsRNA provided for an improvement, when compared to a SNP-targeting dsRNA prior in the art, being more specific in targeting the mutant ataxin-3 gene. When delivered in the striatum of a lentiviral-based mouse model of MJD, via AAV9-mediated expression in a miRNA cassette, was capable of reducing neuronal cell death and mutant ataxin-3 aggregates. Furthermore, it was able to reduce motor behavior deficits, cerebellar neuropathology and magnetic resonance spectroscopy biomarker deficits in a very severe transgenic mouse model of MJD, when intravenously administered.

DsRNAs according to the disclosure can be provided as a siRNA, a shRNA, a pre-miRNA or pri-miRNA. Such dsRNAs may be delivered to the target cells directly, e.g. via cellular uptake using e.g. transfection methods. Preferably, said delivery is achieved using a gene therapy vector, wherein an expression cassette for the siRNA, shRNA, pre-miRNA or pri-miRNA is included in a vector. This way, cells can be provided with a constant supply of dsRNAs to achieve durable ataxin-3 gene suppression without requiring repeated administration. Preferably, the viral vector of choice is AAV9 or derivatives, since this particular AAV serotype efficiently crosses the BBB, enabling intravenous administration. The AAV9, AAVrh10 or derivatives, such as PHP.B or PHP.eB or PHP.S, are available on https://www.addgene.ordviral-service/aav-prep/.

The current disclosure thus provides for the medical use of dsRNAs according to the disclosure, such as the treatment of MJD, wherein such medical use may also comprise an expression cassette or a viral vector, such as AAV9, capable of expressing the said dsRNA of the disclosure.

The present disclosure relates to a double stranded RNA comprising a first strand of RNA and a second strand of RNA, wherein:
the first strand of RNA and the second strand of RNA are substantially complementary to each other, preferably the first and the second strand of RNA are at least 90% complementary to each other;
the first strand of RNA has a sequence length of at least 19 nucleotides;
the first strand of RNA is at least 86% complementary to SEQ ID NO. 1, 7, 13 or 19;
the first strand of RNA is different from SEQ ID NO. 26; and
a first nucleotide of the first strand of RNA is different from cytosine.

In an embodiment, the first strand of RNA may have a sequence length of at least 19 nucleotides to 23 nucleotides, preferably the first strand of RNA may have a sequence length of 20-22 nucleotides, more preferably and to obtain better results the first strand of RNA may have a sequence length of 21-22 nucleotides, even more preferably and to obtain even better results the first strand of RNA may have a sequence length of 22 nucleotides.

In an embodiment, the first strand of RNA may be 90% identical to SEQ ID NO. 2, 3, 4, 5, 6, 8, 9, 10, 11, 12, 14, 15, 16, 17, 18, 20, 21, 22, 23 or 24; preferably 95% identical to SEQ ID NO. 2, 3, 4, 5, 6, 8, 9, 10, 11, 12, 14, 15, 16, 17, 18, 20, 21, 22, 23 or 24; more preferably 100% identical to SEQ ID NO. 2, 3, 4, 5, 6, 8, 9, 10, 11, 12, 14, 15, 16, 17, 18, 20, 21, 22, 23 or 24.

The identity was determined as summarized in the following table:

| | Identity between two sequences (in percentage) and number of | | |
|---|---|---|---|
| Length of the two sequences | sequentially identical bases | | |
| to be compared (nt) | 90% identical | 95% identical | 100% identical |
| 23 nt | 21 | 22 | 23 |
| 22 nt | 20 | 21 | 22 |
| 21 nt | 19 | 20 | 21 |
| 20 nt | 18 | 19 | 20 |
| 19 nt | 17 | 18 | 19 |

In an embodiment, the first strand of RNA may be at least 90% complementary to SEQ ID NO. 1, 7, 13 or 19, preferably 95% complementary to SEQ ID NO. 1, 7, 13 or 19, more preferably 99% complementary to SEQ ID NO. 1, 7, 13 or 19, even more preferably the first strand of RNA is 100% complementary to SEQ ID NO. 1, 7, 13 or 19.

In an embodiment, the first strand of RNA may be selected from SEQ ID NO. 2, 3, 4, 5, 6, 8, 9, 10, 11, 12, 14, 15, 16, 17, 18, 20, 21, 22, 23 or 24.

In an embodiment, and to obtain better results, the first strand of RNA may be complementary to SEQ ID NO. 1 and the first strand of RNA may be selected from SEQ ID NO. 2, 3, 4, 5 or 6.

In an embodiment, and to obtain even better results, the first strand of RNA may be SEQ ID NO. 2 or may be SEQ ID NO. 3.

In an embodiment, the first strand of RNA may be complementary to SEQ ID NO. 7 and the first strand of RNA may be selected from SEQ ID NO. 8, 9, 10, 11 or 12.

In an embodiment, the first strand of RNA may be complementary to SEQ ID NO. 13 and the first strand of RNA may be selected from SEQ ID NO. 14, 15, 16, 17 or 18.

In an embodiment, the first strand of RNA may be complementary to SEQ ID NO. 19 and the first strand of RNA may be selected from SEQ ID NO. 20, 21, 22, 23 or 24.

In an embodiment, and to obtain even better results, the first nucleotide of the first strand of RNA may be a uracil.

In an embodiment, the double stranded RNA may be comprised in a pre-miRNA scaffold, a pri-miRNA scaffold, a miRNA scaffold, a shRNA or a siRNA, preferably a miRNA scaffold or a shRNA, more preferably a miRNA.

In an embodiment, the double stranded RNA may be comprised in a miRNA scaffold, preferably derived from miR-155, such as the one disclosed by Chung et al. (2006), more preferably wherein miR155-based scaffold comprises SEQ ID NO. 27, 28 and 29.

The present disclosure also relates to: an isolated DNA sequence encoding the double stranded RNA now disclosed, an expression cassette comprising said isolated DNA sequence or said double stranded RNA.

This disclosure also relates to a vector comprising the isolated DNA or the double stranded RNA or the expression cassette, now disclosed; preferably wherein said vector is an adeno-associated viral vector or a lentiviral vector or an adenoviral vector or a non-viral vector; more preferably wherein the adeno-associated viral vector is AAV9 or AAVrh10 or PHP.B or PHP.eB or PHP.S.

This disclosure also relates to a host cell comprising the isolated DNA sequence or the double stranded RNA or the expression cassette or the vector now disclosed, preferably wherein said host cell is a eukaryotic cell, more preferably wherein said host cell is a mammalian cell.

This disclosure further relates to a composition comprising the isolated DNA or the double stranded RNA or the expression cassette or the vector or the host cell now disclosed.

This disclosure further relates to a kit comprising the isolated DNA sequence or the double stranded RNA or the expression cassette or the vector or the host cell or the composition now disclosed.

Moreover, the present disclosure also relates to the double stranded RNA, a vector comprising the isolated DNA sequence encoding said double stranded RNA or an expression cassette comprising said isolated DNA sequence, for use in medicine.

The present disclosure further relates to the double stranded RNA, a vector comprising the isolated DNA sequence encoding said double stranded RNA or an expression cassette comprising said isolated DNA sequence, for use in the treatment or in the prevention of a neurodegenerative disease or in the treatment or in the prevention of cytotoxic effects of said neurodegenerative disease, preferably wherein the neurodegenerative disease may be a trinucleotide-repeat disease, more preferably wherein the neurodegenerative disease may be a CAG trinucleotide-repeat disease, even more preferably the double stranded RNA, the vector or expression cassette is administrated to regulate the levels of neurometabolites, preferably to increase N-acetylaspartate, to decrease myo-inositol, glycerophosphocholine and phosphocholine.

In an embodiment, the neurodegenerative disease is the Machado-Joseph disease.

In an embodiment, the double stranded RNA is administrated systemically, intravenously, intratumorally, orally, intranasally, intraperitoneally, intramuscularly, intravertebrally, intracerebrally, intracerebroventriculally, intracisternally, intrathecally, intraocularly, intracardiacally, intradermally, or subcutaneously, preferably intravenously, intracisternally, intrathecally or, in situ, by intracerebral administration.

In the present disclosure, the term complementary means nucleotides of a nucleic acid sequence that can bind to another nucleic acid sequence through hydrogen bonds, i.e. nucleotides that are capable of base pairing. Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A).

In the present disclosure, the term substantial complementarity means that is not required to have the first and second RNA sequence to be fully complementary, or to have the first RNA sequence and SEQ ID NO. 1, 7, 13 or 19 fully complementary.

Furthermore, in the present disclosure, the substantial complementarity between the first RNA sequence and SEQ ID NO. 1, 7, 13 or 19 means having no mismatches, one mismatched nucleotide, two mismatched nucleotides or three mismatched nucleotides. For example, considering the first RNA sequence and SEQ ID NO. 1, it is understood that one mismatched nucleotide means that over the entire length of the first RNA sequence that base pairs with SEQ ID NO. 1 one nucleotide does not base pair with SEQ ID NO. 1. Having no mismatches means that all nucleotides base pair with SEQ ID NO. 1. Having 2 mismatches means two nucleotides do not base pair with SEQ ID NO. 1. Having 3 mismatches means three nucleotides do not base pair with SEQ ID NO. 1. The same applies for the first RNA sequence and SEQ ID NO. 7, first RNA sequence and SEQ ID NO. 13 or first RNA sequence and SEQ ID NO. 19.

In the present disclosure, the first RNA sequence may also be longer than 19 nucleotides; in this scenario, the substantial complementarity is determined over the entire length of SEQ ID NO. 1. This means that SEQ ID NO. 1 in this embodiment has either no, one or two mismatches over its entire length when base paired with the first RNA sequence. The following table illustrates that was explained in the above paragraphs:

| | Pairing between the first strand of RNA and SEQ ID NO. 1, 7, 13 | | | |
|---|---|---|---|---|
| Length of the first | or 19 (in percentage) and number of mismatched nucleotides | | | |
| strand of RNA (nt) | 86% | 90% | 95% | 100% |
| 23 | 3 | 2 | 1 | 0 |
| 22 | 3 | 2 | 1 | 0 |
| 21 | 3 | 2 | 1 | 0 |
| 20 | 3 | 2 | 1 | 0 |
| 19 | 3 | 2 | 1 | 0 |

### BRIEF DESCRIPTION OF THE DRAWINGS

The following figures provide preferred embodiments for illustrating the description and should not be seen as limiting the scope of disclosure.

FIG. 1: Schematic representation of the MJD1 gene and exonic single nucleotide polymorphisms rs1048755 and rs12895357. MJD1 gene is composed by 11 exons (gray boxes). The CAG repeat is located on exon 10 and MJD may be caused by more than 51 repetitions. A SNP was identified immediately after the CAG expansion (nucleotide 987)—r512895357. Non-mutant alleles typically exhibit a guanine (G) in this position, whereas mutant alleles present a cytosine (C) in 70% of MJD patients. Another SNP was identified on exon 8 (nucleotide 669)—r51048755. In this case, non-mutant alleles normally exhibit a guanine (G) in this position, whereas mutant alleles present an adenine (A) in 70% of MJD patients.

FIG. 2: miR-ATXN3 mediates an efficient and allele-specific silencing of mutant ataxin-3 in vitro. (a) Representation of artificial microRNAs (miRs) and short-hairpin (sh) vector constructs. An artificial microRNA construct was designed, based on the silencing sequence SEQ. ID NO. 2 now disclosed, for specifically silencing of mutant ataxin-3 (miR-ATXN3). A control miRNA (miR-control), whose sequence does not silence any mammalian RNA, was also designed. Both were inserted in an AAV2 plasmid vector backbone, under the control of the U6 promoter and with EGFP reporter gene. A plasmid encoding a shRNA that specifically target the mutant ataxin-3, and known in the art (Alves et al., 2008a), was also used (sh-mutATXN3); b, c) Neuro2a cells (mouse neural crest-derived cell line) previously infected with lentiviral vectors encoding for human mutant ataxin-3 with 72Q (b) or human wild-type ataxin-3 with 27Q (c) were transfected with plasmids encoding miR-Control (control condition), miR-ATXN3 and sh-ATXN3. miR-ATXN3 induced a reduction of 42.03±6.26% of human mutant ataxin-3 mRNA levels, not affecting wild-type mRNA levels of human ataxin-3. These results were supported by western blotting in d) and e), respectively. Data represent mean±s.e.m.; NS P>0.05, *P<0.05, and **P<0.01. b, c, d, e) One-way analysis of variance (ANOVA) with Bonferroni's post-hoc test. miR-Control n=5; miR-ATXN3 n=5; sh-ATXN3 n=5. Internal controls for normalization were selected according to GenEx analysis, corresponding to endogenous mouse ataxin-3 and Glyceraldehyde 3-phosphate dehydrogenase (GAPDH) mRNA levels. CMV, Cytomegalovirus enhancer; CBA, Chicken beta-actin promoter; EGFP, Enhanced-green fluorescent protein; ITR, Inverted terminal repeats.

FIG. 3: SEQ ID NO. 3, similarly to SEQ ID NO. 2 (miR-ATXN3) mediates an efficient and allele-specific silencing of mutant ataxin-3 in vitro. a, b) Neuro2a cells previously infected with lentiviral vectors encoding for human mutant ataxin-3 with 72Q (a) or human wild-type ataxin-3 with 27Q (b) were transfected with plasmids encoding miR-Control, SEQ ID NO. 2 (miR-ATXN3), SEQ ID NO.3 or sh-ATXN3. An artificial miR155-based construct encoding SEQ ID NO. 3 induced a reduction of human mutant ataxin-3 mRNA levels similar to a construct encoding SEQ ID NO. 2 (miR-ATXN3), not affecting wild-type mRNA levels. Data represent mean±s.e.m.; NS P>0.05, *P<0.05, **P<0.01, and ***P<0.001. (a, b) One-way analysis of variance (ANOVA) with Bonferroni's post-hoc test. miR-Control n=5; miR-ATXN3 n=5; SEQ ID NO. 3 n=5, and sh-ATXN3 n=5. Internal controls for normalization were selected according to GenEx analysis, corresponding to endogenous mouse ataxin-3 and Glyceraldehyde 3-phosphate dehydrogenase (GAPDH) mRNA levels.

FIG. 4: miR-ATXN3 treatment does not induce alterations in endogenous mouse ataxin-3 mRNA levels in vitro. (a,b) Neuro2a cells infected with (a) human mutant ataxin-3 (72Q) or (b) human wild-type ataxin-3 (27Q) were transfected with plasmids encoding miR-Control, miR-ATXN3 and sh-ATXN3. Relative expression levels of mouse ataxin-3 mRNA were determined by quantitative reverse transcriptase-PCR. Data represent mean relative mRNA levels±s.e.m.; ns=p>0.05 compared with miR-Control. One-way analysis of variance (ANOVA) with Bonferroni's post-hoc test. miR-Control n=5; miR-ATXN3 n=5; sh-ATXN3 n=5.

FIG. 5: miR-ATXN3 reduces the levels of mutant ataxin-3 mRNA and mutant aggregated ataxin-3 and prevents striatal degeneration upon intracranial injection in a lentiviral-based mouse model of MJD. (a) Schematic representation of the strategy used to generate a striatal lentiviral-based mouse model of MJD and to silence mutant ataxin-3 using AAV9. Ten-week-old mice were bilaterally co-injected in the striatum with lentiviral vectors encoding human mutant ataxin-3 with 72Q (LV-Atx3-MUT) and AAV9 vectors encoding miR-ATXN3 in the right hemisphere (AAV9-miR-ATXN3) and miR-Control in the left hemisphere (AAV9-miR-Control). Five weeks after the surgery, mice were euthanized. (b) Image from confocal microscopy showing an effective transduction of mouse striatum of the striatal lentiviral model of MJD by both rAAV9 vectors. (c) Quantitative reverse transcriptase—PCR analysis demonstrated that miR-ATXN3 induced a 63.75±2.25% decrease in the levels of mutant ataxin-3 mRNA, in comparison with left control hemisphere. (d) Western blot analysis also confirmed that miR-ATXN3 expression significantly reduced mutant ataxin-3 aggregates. (e) Ubiquitin immunoreactivity in the striatum of striatal lentiviral-based mouse model of MJD co-injected with miR-Control or mir-ATXN3. Total number of mutant ataxin-3 inclusions were counted and quantified in (f). Scale bar, 50 μm. (g) DARPP-32 staining revealed a major loss of DARPP-32 immunoreactivity in the striatal hemisphere co-infected with human mutant ataxin-3 and miR-Control. Scale bar, 200 μm. This was quantified in (h), as depleted volume of DARPP-32 staining. (i) Cresyl violet staining indicating pycnotic nuclei in both hemispheres. A higher number of pycnotic nuclei were visible in the control hemisphere. This was quantified in j). Scale bar, 20 μm. Data represent mean±s.e.m.; ns p>0.05, *p<0.05, ***p<0.001 compared with control hemisphere. (c,d) Paired Student's t-test. n=5. (f,h) Paired Student's t-test. n=8. (j) Paired Student's t-test. n=4.

FIG. 6: Intravenously injected rAAV9 vectors mediate an efficient transduction throughout the brain of wild-type and transgenic MJD mice. Representative images of GFP immunohistochemistry (in gray) in the brains of 3-month-old mice: A) a non-injected transgenic mouse; B) a transgenic mouse subjected to rAAV9-miR-ATXN3 IV injection at postnatal day 1; C) a wild-type mouse subjected to the same procedure. Images show rAAV9 transduction of the whole brain, cerebellum (CB), hippocampus (HIP), pontine nuclei (PN) and medulla/spinal cord (Md/SC), obtained with 5× and 20× objectives.

FIG. 7: rAAV9 vectors exhibit an efficient transduction of transgenic mouse cerebella. Representative images of GFP visible immunohistochemistry (in gray) in the cerebellum of a 3-month-old mouse subjected to rAAV9-miR-ATXN3 neonatal IV injection. Images were obtained with a 20× objective and show cerebellar regions with particularly efficient transduction including: deep cerebellar nuclei (DCN), lobules 10, 9, 7 and 6 and choroid plexus cells of the fourth ventricle (4V).

FIG. 8: rAAV9 targets the main regions of mutant ataxin-3 accumulation in transgenic mouse cerebella. Representative images showing immunofluorescence for HA and GFP in the cerebellum of a transgenic mouse subjected to rAAV9-miR-ATXN3 injection at P1. Images were obtained in a confocal microscope with a 20× objective. a) Representative image of rAAV9-positive Purkinje cells, showing co-localization of HA and GFP signals (white color). DCN—deep cerebellar nuclei; PCL—Purkinje cell layer.

FIG. 9: Silencing mutant ataxin-3 improves rotarod performance in MJD transgenic mice. a) Experimental plan in MJD transgenic mice, divided into three important tasks: 1) AAV9 intravenous injection at PN1; 2) Behavioral assessment at 3 different time points and 3) Sacrifice and neuropathological analysis. b) Rotarod performance at constant velocity (5 r.p.m). c) Rotarod performance at accelerated velocity. Data are presented as mean latency time to fall±SEM for control mice (miR-Control, n=11) and mice injected with miR-ATXN3 (n=8). Statistical analysis was performed using the unpaired Student's t-test (*P0.05, **p<0.01).

FIG. 10: miR-ATXN3 treatment improves swimming, beam-walking performances and gait ataxia in MJD transgenic mice. a) Animals were evaluated based on the time they took to swim across a pool and climb the platform. Data are presented as mean latency time±SEM. b) Animals were evaluated based on their performance when walking on a 9-mm round beam. Considering the total time to cross the beam and the motor coordination, each animal received a score. Gait pattern was analyzed by measuring: c) hind base width, d) front base width and e) footprint overlap (cm). Data are presented as mean performance score±SEM. Statistical analysis was performed using the unpaired Student's t-test (*p<0.05), comparing the performance of control mice (miR-Control, n=11) and mice injected with rAAV9-miR-ATXN3 (n=8).

FIG. 11: miR-ATXN3 treatment efficiently reduces the number of mutant ataxin-3 aggregates and efficiently preserves molecular layer thickness. a) Representative images of immunofluorescence labeling mutant ataxin-3 (HA in white) in the lobule 10 of control (miR-Control) and treated (miR-ATXN3) transgenic mice. Images were obtained in a confocal microscope with a 20× objective. b) Quantification of mutant ataxin-3 aggregates per area in lobules 10, 9 and 6. c) Representative images of cresyl violet staining in the lobule 10 of treated and control transgenic mice, obtained with a 20× objective. d) Quantification of molecular layer thickness in lobules 10, 9 and 6. Values correspond to the mean±SEM of three specific sections for each animal (miR-Control, n=11; miR-ATXN3, n=8). Statistical analysis was performed using the unpaired Student's t-test (*p<0.05, **p<0.01, ***p<0.001). ML—molecular layer thickness; Lob10—Lobule 10

FIG. 12: Schematic representation of possible mechanisms underlying AAV9-miR-ATXN3 therapeutic impact in the present disclosure. i) rAAV9 vectors encoding miR-ATXN3 were intravenously injected into neonatal MJD transgenic mice, resulting in ii) mutant ataxin-3 silencing in the cerebellum and consequently iii) alleviation of neuropathological and behavioral impairments. Although rAAV9 vectors have efficiently transduced Purkinje cells (PCs) in lobules 10 and 9, other mechanisms could potentially increase their transduction levels and/or beneficial effects, such as: 1) Transfer of viral vectors from the blood to the CSF and/or secretion of miR constructs to the CSF by transduced epithelial cells in the choroid plexus; 2) rAAV9 retrograde transport from DCN to PC layer and/or transfer of miRs from transduced cells in the DCN to PC projections; 3) Transfer or miRs between neighbor PCs; 4) Neuroprotective effects induced by rAAV9-positive PCs. CSF—cerebrospinal fluid; DCN—Deep Cerebellar Nuclei; PC—Purkinje cell

FIG. 13: Different rAAV9 transduction levels correlate with neuropathological and behavioral parameters in treated mice. a) Linear regression graph between GFP mean intensity in lobules 9 and 10 (A.U.=arbitrary units) and number of aggregates/mm² in the same region for rAAV9-miR-ATXN3 treated animals (n=8) (p=0.0309, R²=0.5675). b) Linear regression graph between GFP integrated intensity in all cerebellar lobules (A.U.=arbitrary units) and mean latency to fall in accelerated rotarod for rAAV9-miR-ATXN3 treated animals (n=8), considering all time points (35, 55 and 85 days). According to residual analysis, one animal was considered an outlier for the predicted linear regression model. Analysis was performed without this animal (p=0.0123, R²=0.7457). Statistical analysis was performed using Pearson's correlation (two-tailed p value). Dotted lines represent the 95% confidence intervals.

FIG. 14: rAAV9-miR-ATXN3 IV injection does not affect rotarod performance in wild-type mice. a) Rotarod performance at constant velocity (5 r.p.m). b) Rotarod performance at accelerated velocity. Data are presented as mean latency time to fall±_SEM for wild-type mice (miR-Control, n=5) and mice injected with AAV9-miR-ATXN3 (n=5). Statistical analysis was performed using the unpaired Student's t-test (ns=not significant).

FIG. 15: miR-ATXN3 treatment ameliorates the levels of key metabolites in the cerebellum. a) Magnetic resonance spectroscopy: Cerebellar neurochemical profiles of the miR-control, miR-ATXN3 and WT mice at 75 days. NAA, tChol, and Ins metabolites were highly deregulated in transgenic MJD when compared to WT mice. Mice injected with rAAV9 miR-ATXN3 presented higher levels of NAA (neuronal marker) and lower levels of Ins and tCho (markers of cell death) when compared to control mice, demonstrating the efficacy of the miR-ATXN3 treatment. b) NAA/Ins, NAA/tCho and NAA/(Ins+tCho) ratios were used to evaluate the efficacy of this gene-based therapy. All values are presented as mean±SEM and statistical analysis was performed using the One-way ANOVA. miR-Control (n=8), miR-ATXN3 (n=7), and WT (n=8). Asterisks indicate a statistically significant difference between groups, *p<0.05, ****p<0.0001. Ins: myo-inositol, NAA: N-acetylaspartate, tCho: glycerophosphocholine+phosphocholine.

FIG. 16: Example of a dsRNA of the present disclosure targeting ataxin-3 mRNA at rs12895357 (Cytosine) embedded in an artificial miRNA scaffold using pri-miR-155. First RNA sequence/strand of the dsRNA (SEQ ID NO. 2) is depicted in the rectangle.

### DETAILED DESCRIPTION

The present disclosure provides for a SNP-targeting dsRNA comprising a first RNA sequence/strand and a second RNA sequence/strand, wherein the first and second RNA sequences/strands are substantially complementary to each other, preferably the first strand of RNA and the second strand of RNA are at least 90% complementary to each other, wherein the first RNA sequence/strand has a sequence length of at least 19 nucleotides, preferably has a sequence of 19-23 nucleotides, is at least 86% complementary to SEQ ID NO. 1, 7, 13 or 19. Preferably, the first strand of RNA is different from SEQ ID NO. 26; and a first nucleotide of the first strand of RNA is different from cytosine.

In the present disclosure, to increase the efficiency of gene silencing in mammalian cells, all designed SNP-targeting dsRNAs, without exception, include: i) one uracil (U) at the 5′ end, ii) at least five A/U residues in the first eight nucleotides of the 5′ end terminal and iii) the absence of any GC stretch of more than five nucleotides in length in the first strand (anti-sense strand).

The allele-specific gene silencing now disclosed is achieved by a precise pairing outside the seed region of the first RNA sequence/strand (i.e. anti-sense), more precisely at the position 12, close to the cleavage site. The 5′-terminal ‘seed’ sequence of anti-sense (positions 2-8) is complementary to both alleles (i.e. normal and mutant allele). Therefore, all selected SNP-targeting dsRNAs are fully complementary to the mRNA containing the target SNP allele, but form a mismatch at position 12 with the non-target mRNA, allowing discriminatory silencing.

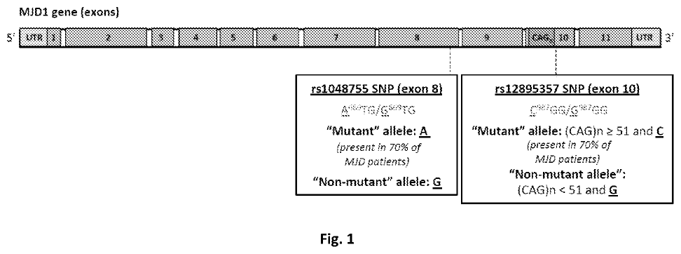

Following this rationale, a silencing sequence (SEQ ID NO. 2) was firstly designed to target cytosine (C) in the SNP located at the 3′ end of the expanded CAG tract of exon 10 of the ataxin-3 gene (C⁹⁸⁷GG/G⁹⁸⁷GG: r512895357). Exon 10 of ataxin-3 gene has over 51 CAG repeats when mutated and a C nucleotide after the over-expanded CAGs in 70% of MJD patients, while non-mutant ataxin-3 allele has typically a G at this position (FIG. 1). This allows SEQ ID NO. 2, as well as SEQ ID NO. 3, 4, 5, or 6, to promote allele-specific silencing of mutant ataxin-3. SEQ ID NO. 8, 9, 10, 11, or 12, can be applied in rare cases where a G nucleotide is present at this position and associated with mutant allele. Moreover, any silencing sequence that targets a C at the r512895357 (SEQ ID NO. 2, 3, 4, 5, or 6) in combination with a silencing that targets a G at this position (SEQ ID NO. 8, 9, 10, 11, or 12), can silence both mutant and non-mutant ataxin-3, leading to a complete knock-down of ataxin-3 expression.

Following the same rationale, the exonic SNP r51048755 (A⁶⁶⁹TG/G⁶⁶⁹TG), located at exon 8, can be also used for allele-specific silencing of mutant ataxin-3 genes (FIG. 1). For instance, SEQ ID NO. 14, 15, 16, 17, or 18 targets an adenine (A) at this position, which is also in linkage disequilibrium with the disease-causing expansion in 70% of MJD families, while SEQ ID NO. 20, 21, 22, 23, or 24 can be used in rare situations where a G nucleotide at this position is associated with a mutant allele.

To evaluate the rationale used in the present disclosure for the design of allele-specific silencing sequences, we firstly conducted in vitro studies to evaluate SEQ ID NO, 2, Thereafter, the therapeutic potential of SEQ ID NO.2 was tested in two different mouse models of MJD, i.e. in a lentiviral-based and in a transgenic mouse model of MJD.

### In Vitro Studies

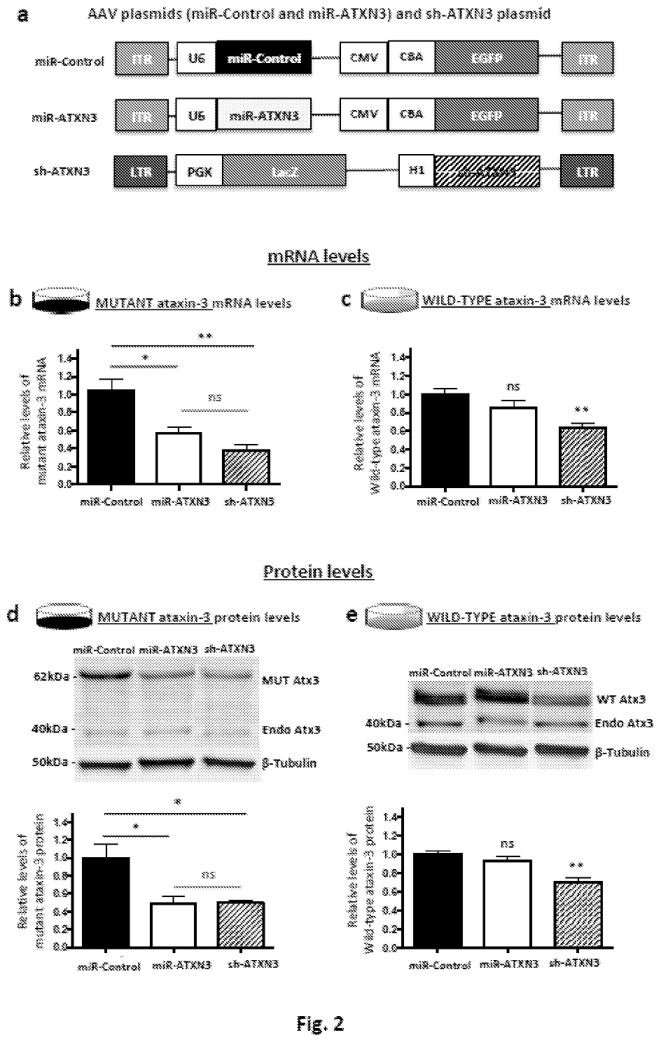

In an embodiment, a miRNA-based RNAi plasmid was produced as follows. Based on the SEQ ID NO. 2 or SEQ ID NO.3, miR155-based artificial miRNAs targeting ataxin-3 mRNA at r512895357 (miR-ATXN3) were designed. A control miRNA, whose sequence does not silence any mammalian mRNA was also designed (miR-Control). Both artificial miRNAs were subsequently cloned into a self-complementary adeno-associated virus serotype 2 backbone (scAAV2-U6-miRempty-CBA-eGFP plasmid), kindly provided by Miguel Sena-Esteves (UMass Medical School, Gene Therapy Center, Worcester, Mass., USA), which include the enhanced green fluorescent reporter gene (EGFP) and where the artificial miRNA is driven by U6 promoter (FIG. 2A).

In an embodiment, a plasmid encoding a shRNA that specifically targets the mutant ataxin-3 and is known in the art (sh-ATXN3) was produced as already described (Alves et al., 2008a) (FIG. 2A). Similarly, to SEQ ID NO. 2, sh-ATXN3 (SEQ ID NO. 26) aims to target a C nucleotide in the SNP located at the 3′ end of the expanded CAG tract of exon 10 (r512895357). The shRNA expression is driven by H1 promoter.

In an embodiment, lentiviral vectors encoding human wild-type (LV-WT-ATXN3) and mutant ataxin-3 (LV-Mut-ATXN3), with 27Q and 72Q respectively, have previously been generated in HEK293T cells with a four-plasmid system, as already described (Alves et al., 2008b). The lentiviral particles were resuspended in 1% bovine serum albumin (BSA) in phosphate-buffered saline (PBS). The viral particle content of batches was determined by assessing HIV-1 p24 antigen levels (RETROtek, Gentaur, Paris, France). Viral stocks were stored at −80° C. until use.

In an embodiment, mouse neural crest-derived cell line (Neuro2a cells) culture was obtained as follows. Mouse neural crest-derived cell line were obtained from the American Type Culture Collection cell biology bank (CCL-131) and maintained in DMEM medium supplemented with 10% fetal bovine serum, 100 U/ml penicillin and 100 mg/ml streptomycin (Gibco) (complete medium) at 37° C. in 5% CO₂/air atmosphere.

In an embodiment, Neuro2a cells infection was carried out as follows. To obtain neuronal cell lines stably expressing mutant or non-mutant (i.e. wild-type) ataxin-3, Neuro2a cells were infected with lentiviral vectors encoding for full-length human mutant ataxin-3 (72Q) with a C at the r512895357 (exon 10), or the wild-type form (27Q) with a G at the same SNP, as previously described. Briefly, Neuro2a cells were incubated with the respective vectors at the ratio of 10 ng of p24 antigen/10⁵ cells, in the presence of polybrene.

In an embodiment, Neuro2a cells transfection was performed as follows. On the day before transfection, Neuro2a cells previously infected with mutant or wild-type ataxin-3 using lentiviral vectors were plated in a twelve-well plate (180.000 cells/well). Cells were transfected with the respective AAV plasmids: miR-Control, miR-ATXN3 and sh-ATXN3, using Polyethylenimine (PEI) linear, Mw 40,000 (Polysciences, Inc., Warrington, Pa., USA), as transfection reagent. Briefly, DNA:PEI complex formation was induced by mixing 10 μL of DMEM, 4 μL of PEI (1 mg/ml) and 800 ng of DNA. Following a 10-minute incubation at room temperature, 500 μL of DMEM complete medium were added to the mixture. Finally, Neuro2a cells were incubated with 500 μL of transfection solution per well, after removing half of the medium. Forty-eight hours after transfection, Neuro2a cells were washed with PBS1×, treated with trypsin, collected by centrifugation and stored at −80° C.

In an embodiment, RNA extraction, DNase treatment and cDNA synthesis were carried out as follows. Total RNA was isolated using Nucleospin RNA Kit (Macherey Nagel, Düren, Germany) according to the manufacturer's instructions. Briefly, after cell lysis, the total RNA was adsorbed to a silica matrix, washed with the recommended buffers and eluted with RNase-free water by centrifugation. Total amount of RNA was quantified by optical density (OD) using a Nanodrop 2000 Spectrophotometer (Thermo Scientific, Waltham, USA) and the purity was evaluated by measuring the ratio of OD at 260 and 280 nm.

In an embodiment, in order to avoid genomic DNA contamination and co-amplification, DNase treatment was performed using Qiagen RNase-Free DNase Set (Qiagen, Hilden, Germany), according to the manufacturer's instructions. Briefly, the final volume of reaction was 6 μL, containing 0.6 μL of DNase buffer, 0.25 μL of DNase and 500 ng of RNA. After a 30-minute incubation at 37° C., 0.5 μL of 20 mM EDTA pH=8 were added to stop the reaction. The final step was a 65° C. incubation for 10 minutes.

In an embodiment, cDNA was then obtained by conversion of 420 ng of total RNA using the iScript Select cDNA Synthesis Kit (Bio-Rad, Hercules, USA) according to the manufacturer's instructions. The complete mix, with a total volume of 10 μL, was prepared using 2 μL of reaction mix (5×), 0.5 μL of iScript reverse transcriptase and the appropriate volume of RNA template and nuclease-free water. The complete reaction mix was incubated 5 minutes at 25° C., followed by 30 minutes at 42° C. and 5 minutes at 85° C. After reverse transcriptase reaction, the mixtures were stored at −20° C.

In an embodiment, quantitative real-time PCR (qPCR) was performed as follows. All qPCRs were performed in an Applied Biosystems StepOnePlus Real-Time PCR system (Life technologies, USA) using 96-well microtiter plates and the SsoAdvanced SYBR Green Supermix (Bio-Rad, Hercules, USA), according to the manufacturer's instructions.

In an embodiment, reactions were performed in a 20 μL of final volume reaction mixture containing 10 μL of SsoAdvanced SYBR Green Supermix (Bio-Rad, Hercules, USA), 10 ng of DNA template and 500 nM of previously validated specific primers for human ataxin-3, mouse ataxin-3, mouse glyceraldehyde 3-phosphate dehydrogenase (GAPDH) and mouse hypoxanthine guanine phosphoribosyl transferase (HPRT) according to MIQE guidelines. The PCR protocol was initiated by a denaturation program (95° C. for 30 seconds), followed by 40 cycles of two steps: denaturation at 95° C. for 5 seconds and annealing/extension at 56° C. for 10 seconds. The melting curve protocol started after amplification cycles, through a gradual temperature increase, from 65 to 95° C., with a heating rate of 0.5° C./55.

In an embodiment, the cycle threshold values (Ct) were determined automatically by the StepOnePlus software (Life technologies, USA). For each gene, standard curves were obtained, and quantitative PCR efficiency was determined by the software. The mRNA relative quantification with respect to control samples was determined by the Pfaff method. Ideal reference genes were determined using the GenEx software.

In an embodiment, protein was extracted from neuro2a cells and homogenized using RIPA lysis buffer mixed with a protease inhibitor cocktail and 2 mM of dithiothreitol. The lysate was further sonicated and protein concentration estimated through the Bradford method (Bio-Rad Protein Assay, Bio-Rad). Sixty micrograms of total denatured protein were then loaded in a 4% stacking, 10% resolving polyacrylamide gel for electrophoretic separation. Proteins were then transferred to polyvinylidene difluoride (PVDF) membranes (Merck Millipore) and blocked in 5% nonfat milk. Immunoblotting was performed using the monoclonal anti-ataxin-3 antibody (1H9, 1:1000; Chemicon), and beta-tubulin. Densitometric quantification of mutant or non-mutant human ataxin-3 and endogenous mouse ataxin-3 was relative to beta-tubulin protein.

### In Vivo Studies

In an embodiment, the production of adeno-associated viral serotype 9 (AAV9) vectors was carried out as follows. Briefly, vector stock was prepared by triple transfection of HEK293T cells with calcium phosphate precipitation of AAV constructs (miR-ATXN3 and miR-Control), pFΔ6 (adenoviral helper plasmid) and AAV9 rep/cap plasmid, as previously described leading to the production of rAAV9-miR-ATXN3 and rAAV9-miR-Control. AAV9 vectors were then purified by iodixanol gradient centrifugation, followed by concentration and dialysis as previously described. The vector titer was determined by quantitative real-time PCR (qPCR) with specific primers and probe for bovine growth hormone polyA element (pBGH).

In an embodiment, it was assessed the functionality and efficacy of this AAV9-based strategy in a lentiviral (LV)-based mouse model of MJD upon intracranial administration (FIG. 5A). This particular mouse model allows testing therapeutic approaches in a short time and quantitative analysis of the neuropathological deficits induced by mutant ataxin-3 expression (Alves et al., 2008b).

In an embodiment, thirteen 10-weeks old mice were anesthetized and co-injected bilaterally in the striatum with lentiviral vectors encoding human mutant ataxin-3 (72Q) (3×10⁵ ng of p24) and rAAV9 vectors encoding an artificial miR targeting mutant ataxin-3 mRNA in the right hemisphere (AAV9-miR-ATAX3) (7×10⁹ viral genomes), and rAAV9 vectors encoding a control miR in the left hemisphere (AAV-miR Control) (7×10⁹ viral genomes) (FIG. 5A).

In an embodiment, western-blot of both hemispheres from three animals was performed. The injected striata were dissected and homogenized using RIPA lysis buffer mixed with a protease inhibitor cocktail and 2 mM of dithiothreitol. The lysate was further sonicated and protein concentration estimated through the Bradford method (Bio-Rad Protein Assay, Bio-Rad). Sixty micrograms of total denatured protein were then loaded in a 4% stacking, 10% resolving polyacrylamide gel for electrophoretic separation. Proteins were then transferred to polyvinylidene difluoride (PVDF) membranes (Merck Millipore) and blocked in 5% nonfat milk. Immunoblotting was performed using the monoclonal anti-ataxin 3 antibody (1H9, 1:1000; Chemicon), and anti-actin (clone AC-74, 1:5000; Sigma). Densitometric quantification of mutant aggregated ataxin-3 was relative to beta-actin protein.

In an embodiment, coronal sections showing complete rostrocaudal sampling of the striatum (12 sections/animal) were scanned using Zeiss Axio Imager Z2 microscope with a ×20 objective. The analyzed areas of the striatum encompassed the entire region ubiquitin inclusions, as revealed by staining with the anti-ubiquitin antibody. All inclusions and their area were counted using an automatic image-analysis software package (Image J software, USA).

In an embodiment, the extent of DARPP-32 loss in the striatum was analyzed by digitizing 12 stained-sections per animal (25 μm thickness sections at 200 μm intervals) to obtain complete rostrocaudal sampling of the striatum. To calculate the DARPP-32 loss, sections were imaged using the tiles feature of the Zen software (Zeiss). The depleted area of the striatum was estimated using the following formula: Volume=d (a1+a2+a3+ . . . ), where d is the distance between serial sections (200 μm) and a1, a2, a3 are DARPP-32-depleted areas for individual serial sections.

In an embodiment, quantitative analysis of the number of condensed pycnotic nuclei in the striatum was performed by analyzing 3 stained-sections per animal (closed to the needle track) at 200 μm intervals. The quantification was performed manually using Adobe Photoshop software.

In an embodiment, polyQ69-transgenic MJD mice were also used. This model expresses N-terminal-truncated human ataxin-3 with a 69 polyglutamine tract specifically in cerebellar Purkinje cells, under the control of L7 promoter. Moreover, the mutant protein exhibits a haemagglutinin (HA) epitope at the amino terminus. Importantly, the transgene contains the previously identified SNP downstream of the CAG expansion (r512895357), therefore showing complementary with miR-ATXN3. Transgenic mice are characterized by an accumulation of mutant ataxin-3 in Purkinje cell layer and deep cerebellar nuclei and pronounced cerebellar atrophy. They exhibit a severe ataxic phenotype starting at postnatal day 21 (P21).

In an embodiment, the transgenic mice colony (C57BL/6 background) was maintained at the animal house facility of the Centre for Neuroscience and Cell Biology of Coimbra (CNC) by backcrossing heterozygous males with C57BL/6 females. Animals were housed in a temperature-controlled room maintained on a 12 h light/12 h dark cycle. Food and water were provided ad libitum. Genotyping was performed by PCR at 4 weeks of age.

In an embodiment, the experiments were carried out in accordance with the European Community Council Directive (86/609/EEC) for the care and use of laboratory animals. The researchers received adequate training (FELASA certified course) and certification to perform the experiments from Portuguese authorities (Direcção Geral de Veterinária).

In an embodiment, experimental design was performed as follows. The present disclosure used 19 female heterozygous MJD mice, injected at postnatal day 1 (P1), with AAV9 encoding miR-ATXN3 (n=8) and AAV9 encoding miR-Control (n=11) (FIG. 9A).

In an embodiment, control and treated MJD mice were then evaluated based on their behavioral performance and neuropathological alterations. A battery of behavioral tests was performed at 35, 55 and 85 days. Mice were sacrificed at postnatal day 95 (P95), followed by brain pathology analysis.

In an embodiment, AAV9 neonatal injection was performed as follows. Intravenous injections were performed in the facial vein of newborn MJD mice and wild-type littermates (P1). In an optimized protocol, firstly the neonates were anesthetized using a bed of ice during approximately 1 minute. After that, a total of 3.5×10¹¹ vg of AAV9 vectors were injected, in a total volume of 50 μL, into the facial vein using a 30-gauge syringe (Hamilton, Reno, Nev., USA). A correct injection was verified by noting blanching of the vein.

In an embodiment, the behavioral testing was performed as follows. MJD transgenic mice performed a battery of behavioral tests at 35, 55 and 85 days of age, in the same dark and quiet room with controlled temperature, after one hour of acclimatization.

In an embodiment, the rotarod apparatus (Letica Scientific Instruments, Panlab) was used in order to evaluate MJD mice motor coordination and balance, by measuring their latency to fall (in seconds). The performance was analyzed at stationary rotarod, using a constant speed of 5 rpm and at accelerated rotarod, in which the velocity gradually increased from 4 to 40 rpm, both for a maximum of 5 minutes. For each time point (35, 55 and 85 days), the test was performed at three consecutive days, with a total of four trials per day. Between subsequent trials, mice had a resting period of at least 20 minutes. For statistical analysis, the mean latency to fall for each time point was calculated considering all consecutive days and trials.

In an embodiment, in order to evaluate possible toxicity due to the treatment, a group of wild-type mice subjected to rAAV9-miR-ATXN3 (n=5) and rAAV9-miR-Control (n=5) IV injection also performed rotarod tests. In this case, the test was performed only in the last time point (85 days) at two consecutive days, with a total of four trials per day. For statistical analysis, the mean latency to fall was calculated considering the second day.

In an embodiment, MJD mice limb coordination was also evaluated through swimming performance in a glass tank (70 cm long, 12.5 cm wide and with 19.5 cm height-walls). The pool presents one visible platform at the end and was filled with water until its level (8.5 cm). Mice were then placed at one end of the tank and were encouraged to swim to the escape platform at the opposite extremity. For each time point, animals performed four trials, swimming across the tank twice per trial and with at least 20 minutes of rest between trials. Their performance was video recorded, in order to measure the time required to swim the whole distance and climb the platform with their four paws. Statistical analysis was based on the mean scores of trials 2, 3 and 4.

In an embodiment, MJD mice motor coordination and balance were assessed by evaluating their ability to cross a series of elevated beams. Long wood beams were placed horizontally, 20 cm above a padded surface with both ends mounted on a support. For each time point, mice performed two consecutive trials on each beam, progressing from the easiest to the most difficult one: i) 18-mm square wide, ii) 9-mm square wide and iii) 9-mm round diameter beams. For all of them, mice had to traverse 40 cm to reach an enclosed safety platform. The latency to cross the beam and the motor performance were recorded and scored according to a predefined rating scale.

In an embodiment, MJD mice footprint patterns were analyzed in order to compare different gait parameters. After coating fore and hind paws with non-toxic red and blue paints respectively, the animals were encouraged to walk in a straight line on a 50 cm long, 10 cm wide, paper-covered corridor into an enclosed box. For each time point, five consecutive steps in each side, preferentially at the middle of the run, were selected for analysis. Stride length values were measured, corresponding to the distance between subsequent left and right forelimbs and hindlimbs. The hind and front base width were determined by measuring the distance between right and left hind and front paws, respectively. In order to assess step alternation uniformity, the overlap was measured as the distance between the fore- and hind-paw from the same side. For each time point, the mean value obtained for the selected five consecutive steps was used for statistical analysis.

In an embodiment, in vivo image acquisition was conducted with a 9.4 T magnetic resonance small animal scanner (BioSpec 94/20) with a standard Bruker cross-coil setup using a volume coil for excitation (86/112 mm of inner/outer diameter, respectively) and a quadrature mouse surface coil for signal detection (Bruker Biospin, Ettlingen, Germany) at the Institute for Nuclear Sciences Applied to Health (ICNAS), University of Coimbra. Volumetric analyses and 1H-MRS were performed.

In an embodiment, tissue preparation was performed after an overdose of pentobarbital, mice were intracardiacally perfused with cold PBS 1× followed by fixation with 4% cold paraformaldehyde (PFA 4%). The brains were then removed and post-fixed in 4% paraformaldehyde for 24 h at 4° C. and cryoprotected by incubation in 25% sucrose/PBS for 48 h at 4° C.

In an embodiment, for each animal, 96 sagittal sections of 30 μm were cut throughout one brain hemisphere using a cryostat (LEICA CM3050S, Germany) at −20° C. They were then collected and stored in two 48-well plates, as free-floating sections in PBS 1× supplemented with 0.05% sodium azide at 4° C.

In an embodiment, the immunohistochemistry protocol was performed as previously reported (Alves et al., 2010). For each animal, eight sagittal sections with an intersection distance of 240 μm were selected.

In an embodiment, the procedure started with endogenous peroxidase inhibition by incubating the sections in PBS1× containing 0.1% Phenylhydrazine (Merck, USA), for 30 minutes at 37° C. Subsequently, tissue blocking and permeabilization were performed in 0.1% Triton X-100 10% NGS (normal goat serum, Gibco) prepared in PBS1×, for 1 hour at room temperature. Sections were then incubated overnight at 4° C. with the primary antibody Rabbit anti-GFP (Invitrogen), previously prepared on blocking solution at the appropriate dilution (1:1000). After three washings, brain slices were incubated in anti-rabbit biotinylated secondary antibody (Vector Laboratories) diluted in blocking solution (1:250), at room temperature for 2 h. Subsequently, free-floating sections were rinsed and treated with Vectastain ABC kit (Vector Laboratories) during 30 minutes at room temperature, inducing the formation of Avidin/Biotinylated peroxidase complexes. The signal was then developed by incubating slices with the peroxidase substrate: 3,3′-diaminobenzidine tetrahydrochloride (DAB Substrate Kit, Vector Laboratories). The reaction was stopped after achieving optimal staining, by washing the sections in PBS1×. Brain sections were subsequently mounted on gelation-coated slides, dehydrated in an ascending ethanol series (75, 95 and 100%), cleared with xylene and finally coverslipped using Eukitt mounting medium (Sigma-Aldrich).

In an embodiment, images of sagittal brain sections subjected to GFP immunohistochemistry were obtained in Zeiss Axio Imager Z2 microscope. Whole-brain images were acquired with an EC Plan-Neofluar 5×/0.16 objective, whereas images of particular regions were obtained with a Plan-Apochromat 20×/0.8 objective.

In an embodiment, immunofluorescence was also performed. For each animal, eight sagittal sections with an intersection distance of 240 μm were selected. Briefly, the protocol started with a blocking and permeabilization step, in which free-floating sections were kept in 0.1% Triton X-100 in PBS1× supplemented with 10% NGS (normal goat serum, Gibco), for 1 h at room temperature. Brain slices were then incubated overnight at 4° C. with the following primary antibodies diluted in blocking solution (10% NGS, 0.1% Triton X-100 in PBS): Mouse anti-HA (1:1000, Invivo Gen) and Rabbit anti-GFP (1:1000, Invitrogen). Following three washing steps in PBS1×, free-floating sections were incubated 2 h at room temperature in fluorophore-coupled secondary antibodies prepared in blocking solution at the appropriate dilution: anti-mouse and anti-rabbit conjugated to Alexa Fluor 594 and 488 (1:200, Life technologies), respectively. After three rising steps in PBS1×, nuclear staining was performed using DAPI (4′,6-diamidino-2-phenylindole). Subsequently, brain sections were washed, mounted on gelatin-coated microscope slides and finally coverslipped on Dako fluorescence mounting medium (S3023).

In an embodiment, cresyl Violet staining was performed using eight sagittal sections with an intersection distance of 240 μm per animal. Selected brain sections were pre-mounted on gelatin-coated slides and dried at room temperature. After being washed in water, sections were subjected to dehydration (using ethanol 96% and 100%), defatting (using xylene substitute) and rehydration (using ethanol 75% and water). Then, slides were immersed in cresyl violet for 5 minutes, in order to stain the Nissl substance present in the neuronal bodies. Finally, sections were washed in water, differentiated in 70% ethanol and dehydrated by passing through 96% and 100% ethanol solutions. Following a clearing step in xylene, sections were mounted with Eukitt (Sigma-Aldrich).

### Immunofluorescence Quantitative Analysis

Following GFP and HA immunofluorescence, specific sagittal sections were selected to acquire images of the whole cerebellum. Serial z-stack images (interval=0.9 μm) were captured by a confocal microscope (Zeiss Cell Observer Spinning Disk Microscope). Images were acquired with a Plan-Apochromat 20×/0.8 objective, using solid state lasers lines (561 nm or 488) for excitation.

In an embodiment, the quantification of mean and integrated GFP fluorescence intensity was performed in 3 specific sagittal sections from treated animals (cut in a sagittal plane 0.48, 0.72 and 0.96 mm lateral to the midline: Sagittal diagrams 105, 107 and 109 in (Franklin and Paxinos)). Images of the whole cerebellum were acquired using confocal microscopy, as already described. Then, maximum intensity projections were obtained for each section, using Zen Black 2012 software.

In an embodiment, mean GFP fluorescence intensity was determined to quantify the viral transduction level in specific cerebellar lobules. For each section, mean GFP fluorescence intensity was determined by the Zen software and calculated after background subtraction. Final values correspond to the average intensity, considering the three analyzed sections per animal.

In an embodiment, integrated GFP fluorescence intensity was determined for cerebellar lobules altogether, in order to compare total viral transduction levels in different animals. In this case, mean GFP fluorescence intensity was determined including all cerebellar lobules and this value was multiplied by the respective area, to calculate integrated fluorescence intensity. Final values correspond to the average integrated intensity, considering the three analyzed sections per animal.

In an embodiment, the quantitative analysis of haemagglutinin-tagged (HA) aggregates was performed as follows. Three specific sections per animal were selected to quantify the number of aggregates in lobules 10, 9 and 6 (sagittal planes 0.48, 0.72 and 0.96 mm lateral to the midline for lobules 9 and 10; sagittal planes 0.72, 0.96 and 1.68 mm lateral to the midline for lobule 6, according to (Franklin and Paxinos)).

Images were acquired using a confocal microscope, as previously described. Average intensity projections were obtained for each section, using Zen Black 2012 software. After manual quantification of the number of aggregates in each lobule, the value was normalized with the respective lobular area, determined in the Zen software. Final values correspond to the average number of aggregates/mm² in the three selected sections per animal. Both treated and control groups were included in this analysis.

In an embodiment, quantification of molecular layer thickness was carried out as follows. Three specific sections per animal were selected to quantify molecular layer thickness in lobules 10, 9 and 6, following cresyl violet staining (sagittal planes 0.48, 0.72 and 0.96 mm lateral to the midline for lobules 9 and 10; sagittal planes 0.72, 0.96 and 1.68 mm lateral to the midline for lobule 6, according to (Franklin and Paxinos)).

In an embodiment, images of the whole cerebellum were obtained in Zeiss Axio Imager Z2 microscope with a Plan-Apochromat 20×/0.8 objective and analyzed with Zen Blue software.

In an embodiment, for each section, molecular layer thickness was calculated separately in lobules 10, 9 and 6, using three measurements in predefined specific regions. Final values correspond to the mean molecular layer thickness in the respective lobule, considering the three selected sections per animal. Both treated and control groups were included in this analysis.

In an embodiment, statistical analysis was performed using Prism GraphPad software. Data are presented as mean±standard error of mean (SEM) and outliers were removed according to Grubb's test (alpha=0.05). Unpaired Student's t-test was performed to compare control and treated groups, whereas One-way ANOVA test was used for multiple comparisons. Correlations between parameters were determined according to Pearson's correlation coefficient. Significance was determined according to the following criteria: p>0.05=not significant (ns); *p<0.05, **p<0.01 ***p<0.001 and ****p<0.0001.

The present disclosure relates to SNP-targeting dsRNAs that can specifically target and reduce the levels of human mutant ataxin-3 protein, while maintaining the levels of the non-mutant form.

The present disclosure relates in particular to a dsRNA sequence (SEQ ID NO. 2) that was designed to precisely target the C nucleotide at the SNP located at the 3′ end of the expanded CAG tract of exon 10 of the ataxin-3 gene (r512895357), which has been reported to be associated with abnormal CAG expansion in 70% of MJD patients worldwide (FIG. 1).

In an embodiment, SEQ ID NO. 2 was incorporated into a miR-155 scaffold, generating an artificial microRNA (FIG. 16). In parallel, a control sequence (SEQ. ID NO. 25), which does not silence any mammalian mRNA, was also used and incorporated into a miR-155 scaffold. Both artificial miRNAs were cloned into self-complementary AAV2 backbones under the control of U6 promoter and with EGFP as reporter gene (miR-Control and miR-ATXN3) (FIG. 2A).

In order to confirm the silencing capacity and specificity of this novel silencing sequence, miR-ATXN3 plasmid was transfected in a mouse neural crest-derived cell line (Neuro2a), previously infected with lentiviral vectors stably expressing: i) human mutant ataxin-3 (72Q) with a C nucleotide at the r512895357 or ii) human wild-type ataxin-3 (27Q) with a G nucleotide at the r512895357. miR-Control plasmid was used as the negative control and a lentiviral plasmid encoding a sh-ATXN3 known in the art to silence human mutant ataxin-3 (SEQ ID NO. 26), was used as a positive control (FIG. 2A).

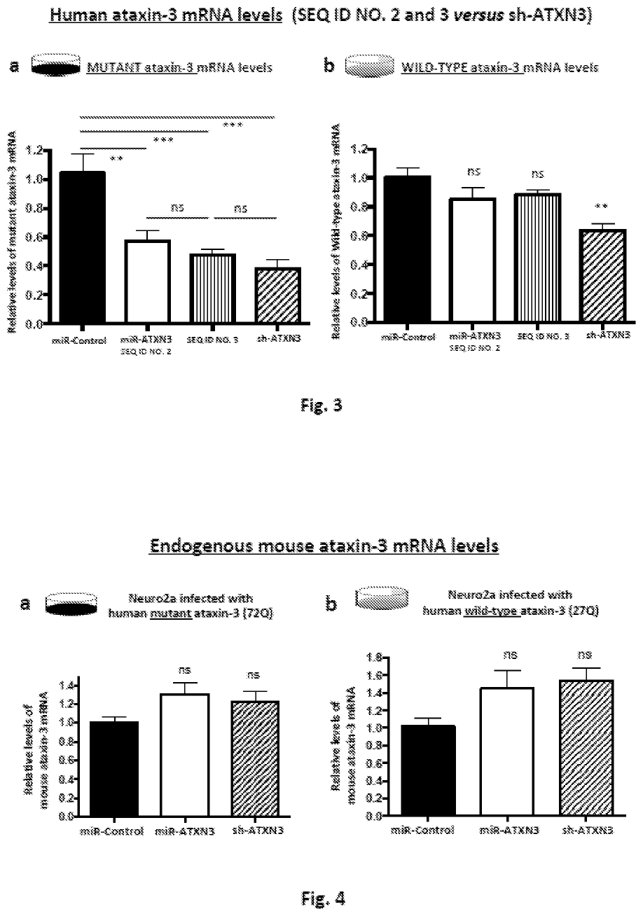

According to quantitative reverse transcriptase-PCR (qPCR) results, transfection with miR-ATXN3 plasmid resulted in a 42,03%±6.26% reduction in mutant ataxin-3 mRNA levels, close to what occurs in the presence of sh-ATXN3 (FIG. 2B). However, in contrast to sh-ATXN3, no alterations were detected in wild-type ataxin-3 mRNA levels after transfection with mut-ATXN3 plasmid (FIG. 2C), proving that SEQ ID NO. 2 precisely targets the C nucleotide at the SNP r512895357 allowing allele-specific silencing of human ataxin-3. Similar results were obtained using an artificial miRNA-155 construct encoding SEQ ID NO. 3, as depicted in FIG. 3.

In terms of protein levels, miR-ATXN3 was as effective as sh-ATXN3 in the reduction of mutant ataxin-3 protein levels (FIG. 2D) (miR-ATXN: 50.66±8.34% versus sh-ATXN3: 55.65±6.04%); however it was much more selective. In fact, no alterations in wild-type protein levels were detected after transfection with miR-ATXN3 plasmid, while sh-ATXN3 induced a significant reduction of human wild-type ataxin-3 mRNA levels in Neuro2a cells expressing the wild-type form (FIG. 2E).

Altogether this indicates that the miR-based strategy now disclosed retains the mutant ataxin-3 silencing ability of previously reported sequences in the art, but that it is much more selective. This means that miR-ATXN3 allows discrimination between mutant and wild-type transcripts, thereby maintaining ataxin-3 normal functions, a significant advantage when translating this therapeutic approach to human patients.

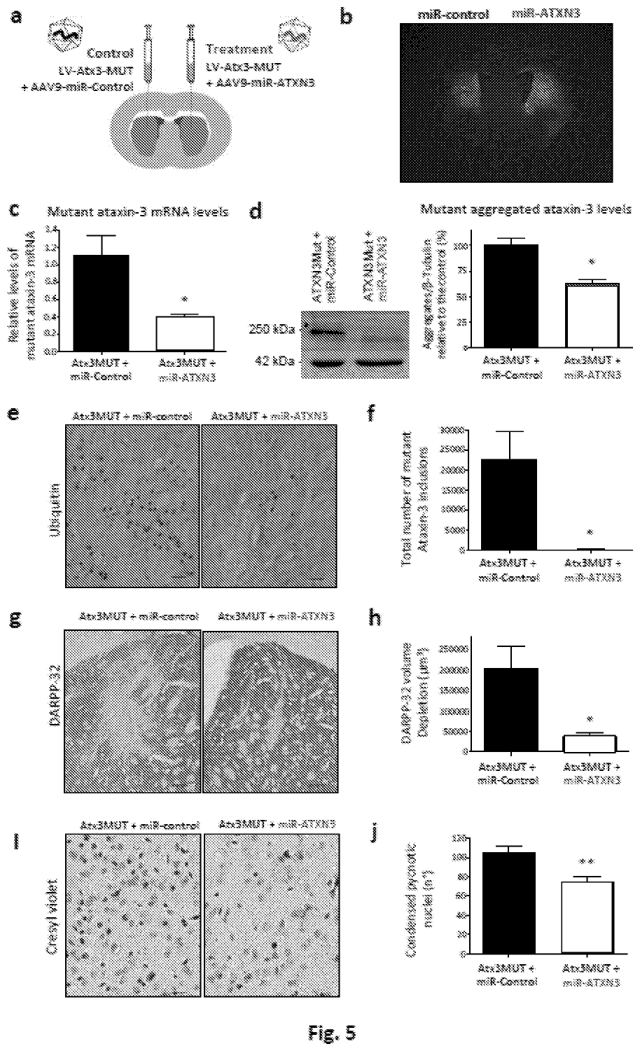

Moreover, no alterations in the levels of endogenous mouse ataxin-3 mRNA were detected (FIG. 4), proving that the silencing effect is specific for human ataxin-3 mRNA.

To explore the therapeutic potential of SEQ ID NO. 2 in vivo, miR-ATXN3 and miR-Control AAV plasmids were packaged into rAAV9 capsids. AAV vector was considered the preferred platform for CNS gene delivery, given its efficient neuronal transduction, long-term transgene expression and safety profile. In particular, AAV serotype 9 (AAV9) has also the capacity to bypass the BBB in wild-type rodents, cats, non-human primates and human, enabling intravenous injection (IV).

miR-ATXN3 was tested in two different mouse models of MJD, i.e. in a lentiviral-based and in a transgenic mouse model of MJD, by intraparenchymal and intravenous administration, respectively.

Firstly, we assessed the functionality and efficacy of miR-ATXN3 in a lentiviral (LV)-based mouse model of MJD upon intracranial (IC) administration. This mouse model allows testing therapeutic approaches in a short time and a precise quantitative analysis of the neuropathological deficits induced by mutant ataxin-3 expression (Alves et al., 2008b). Therefore, thirteen 10-weeks old mice were co-injected bilaterally in the striatum with lentiviral vectors (LVs) encoding human mutant ataxin-3 with 72 CAG repeats (LV-Atx3-MUT) and rAAV9 vectors encoding miR-ATXN3 in the right hemisphere and rAAV9 vectors encoding a miR-Control in the left hemisphere (FIG. 5A).

Five weeks after injection, five mice were sacrificed to evaluate the expression levels of mutant ataxin-3 mRNA (by qPCR) and mutant aggregated ataxin-3 protein levels (by western blotting) and eight mice were perfused and sacrificed for immunohistochemistry analysis (EGFP, anti-ubiquitin, DARPP-32, cresyl violet).

As depicted in FIG. 5B, fluorescence microscopy showed that intracranial administration of AAV9 vectors was effective in both hemispheres, as can be seen by the intense expression of the reporter gene EGFP.

By qPCR (FIG. 5C) and by western blotting (FIG. 5D), it was observed that the expression of mir-ATXN3 induced a 63.75±2.25% decrease in the striatal mRNA levels of mutant ataxin-3 and a 37.64±4.52% decrease in the aggregated form of mutant ataxin 3 respectively, when compared to left control hemisphere.

Since the presence of neuronal intranuclear inclusions containing aggregated ataxin-3 is one important hallmark of MJD, next it was evaluated the potential of miR-ATXN3 treatment to decrease the total number and size of ubiquitin-positive inclusions upon IC administration. When compared to the left control hemisphere, it was observed a clearance of the number of aggregates in the hemisphere injected with rAAV9 encoding miR-ATXN3, demonstrating the efficacy of the treatment (FIGS. 5 E and F).

Then, to evaluate if this strategy could mediate striatal neuroprotection, it was performed an immunohistochemistry against DARPP-32, a regulator of dopamine receptor signaling and a sensitive marker for neuronal dysfunction, that we have previously demonstrated to be a sensitive marker to detect early neuronal dysfunction in the LV-based model of MJD (Alves et al., 2008b). Intracranial administration of miR-ATXN3 decreased the DARP-32 depleted lesion (in 80.87%) when compared to miR-Control (FIGS. 5 G and H).

Finally, cresyl violet staining was performed to evaluate cell injury due to the mutant ataxin-3 expression and a clear reduction of hyperchromatic nuclei was observed in the right-treated hemisphere (approximately 30%) (FIGS. 5 I and J).

Overall, these results show that allele-specific silencing of mutant ataxin-3 based on AAV9-based strategy was effective in reducing the levels of mutant ataxin-3 m RNA and mutant aggregated protein upon IC injection. This promoted the clearance of ubiquitin-positive inclusions, preventing cell injury and striatal degeneration.

Next, we explored the ability of developed rAAV9 vectors to transpose the BBB and to transduce neurons upon intravenous injection in a severely impaired transgenic mouse model of MJD (PolyQ69 transgenic mice) and in their wild-type littermates at P1. This transgenic mouse model expresses a truncated form of human ataxin-3 containing 69 glutamine repeats specifically in the cerebellar Purkinje cells (PCs) and develops a severe and early-onset (P21) pathological phenotype. Moreover, this MJD transgenic mouse model allows the evaluation of allele-specific strategies, as the truncated human ataxin-3 carries the C variant at r512895357 SNP, present in 70% of the MJD patients.

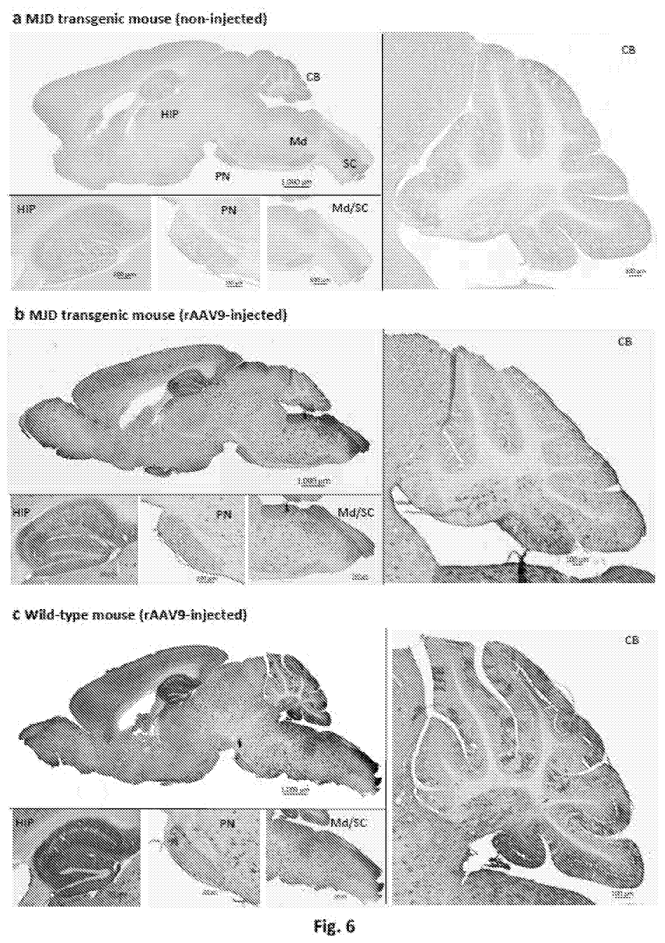

In fact, to get therapeutic impact in PolyQ69 transgenic mice, IV-injected rAAV9 vectors have to circumvent the BBB and efficiently transduce the brain. As a result, the study of rAAV9 distribution in MJD transgenic mouse brain, after sacrifice at 95 days old, was carried out. For that purpose, immunohistochemistry of sagittal brain sections using an antibody against green fluorescent protein (GFP), the reporter gene present in the AAV-plasmids was carried out. Besides analyzing sections from rAAV9-injected transgenic mice, we also used a non-injected MJD mouse as a negative control and a rAAV9-injected wild-type (WT) mouse to compare rAAV9 distribution (FIG. 6).

The pattern of GFP expression was very similar in the transgenic mice, both in the control and treated groups, subjected to rAAV9 IV injection. The vector proved to efficiently spread throughout the brain, including regions normally affected in MJD such as the cerebellum, brainstem, spinal cord and striatum. In particular, the pontine nuclei, which is a major site of degeneration in MJD, showed great transgene expression. Other efficiently transduced areas included the cerebral cortex, olfactory bulb and hippocampus. rAAV9 IV injection into the tail vein of transgenic adult animals, also mediated an effective transduction of mouse brain. The main difference observed between transgenic and wild-type animals corresponds to cerebellar GFP expression. In fact, MJD animals exhibit a weaker and spatially limited GFP signal, when comparing to the robust transgene expression in the whole cerebellum of WT mice (FIGS. 6B and C). These observations might be explained by cerebellar vascularization defects in this particular transgenic animal model, which have already been reported.

Given that human mutant ataxin-3 expression is restricted to the cerebellar PCs of polyQ69 MJD transgenic mice, the therapeutic action of rAAV9-miR-ATXN3 greatly depends on vector ability to transduce the cerebellum, particularly this cellular subtype. Therefore, it was analyzed in further detail the distribution of GFP signal in this region, after immunohistochemical processing.

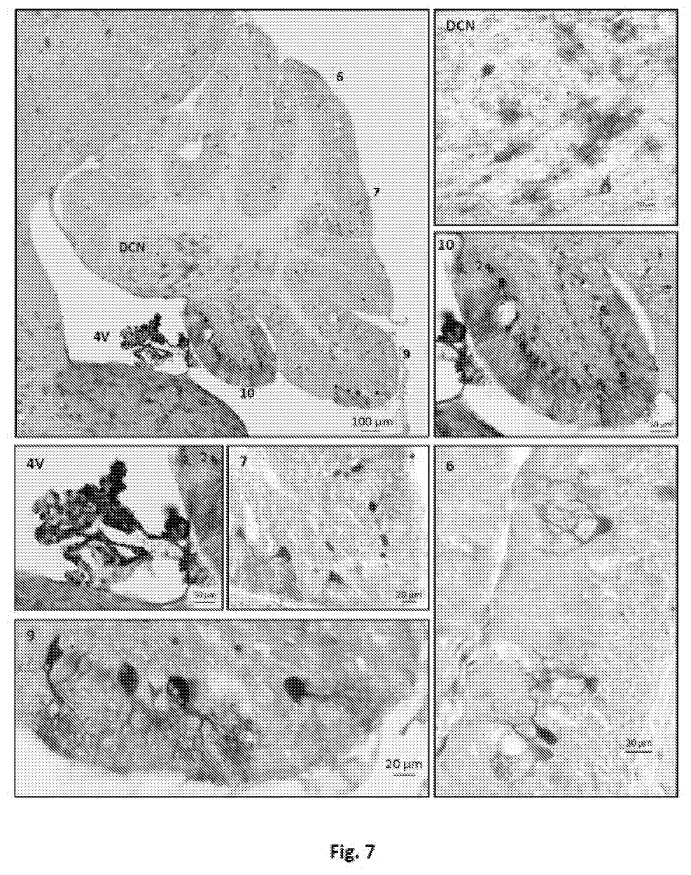

As shown in FIG. 7, GFP expression was not equally distributed throughout the cerebellum, being particularly evident in cerebellar lobule 10, followed by the deep cerebellar nuclei (DCN, probably the most precociously affected region in MJD) and lobule 9. Transduced isolated neurons were also detected in lobules 6 and 7 and in the remaining lobules, although to a less extent. Importantly, choroid plexus cells of fourth ventricle also exhibited a marked GFP expression. This pattern of GFP distribution was observed for all transgenic animals subjected to rAAV9 IV injection, including the control and treated groups.

This preferential cerebellar transduction on lobules 9 and 10 might occur due to a better vascularization in this region or likely due to its proximity with the choroid plexus of the fourth ventricle. The choroid plexus (CP) is composed by a monolayer of epithelial cells, which are responsible for cerebrospinal fluid (CSF) production and constitute a barrier between blood and CSF—the blood-CSF barrier (BCSFB). Therefore, blood-circulating rAAV9 vectors reaching the CP might eventually circumvent the BCSFB and pass to the CSF. Since lobule 10 is close to the CP and in the path of CSF flow, rAAV9 access to PCs would preferentially occur in this cerebellar region.

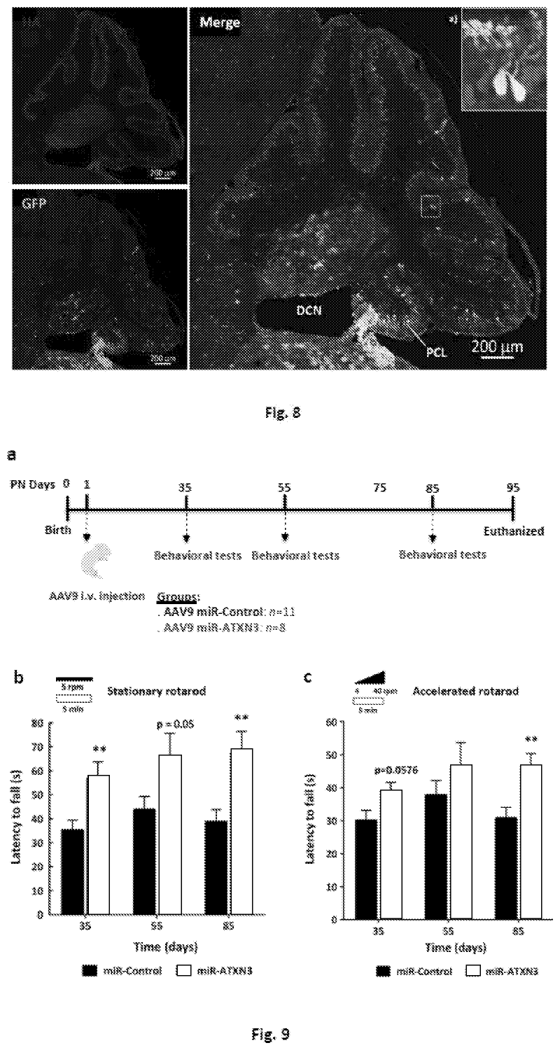

Finally, it was assessed whether rAAV9 targets the cell population mainly affected in this mouse model, i.e. the PCs that express mutant ataxin-3. For that, a co-immunofluorescence labeling both GFP and Haemagglutinin (HA) was performed. As expected, HA signal was detected in the PC cell layer, in which mutant ataxin-3 was distributed throughout the soma with a diffuse staining and in the form of aggregates (FIG. 8). Moreover, mutant ataxin-3 aggregates were also detected in the axon terminals of PCs, in deep cerebellar nuclei (DCN). When comparing the distribution of GFP and HA signals, it was observed a co-localization in the PC layer and in the DCN, the two major regions of mutant ataxin-3 accumulation (FIG. 8). This latter finding indicates that AAV vectors might be retrogradely transported from the DCN to the PCs, contributing to therapeutic impact. Furthermore, DCN rAAV9 transduction might be also beneficial in MJD patients since this region is severely affected in the disease context. This pattern was similar for all rAAV9-IV injected mice, including control and treated groups.

In the present disclosure it was also investigated whether rAAV9-miR ATXN3 injection would alleviate MJD-associated behavioral deficits. The most common MJD symptoms include impairments in motor coordination and balance, as well as ataxic gait. PolyQ69 transgenic mice successfully mimic these features, showing an extremely severe ataxic phenotype with an early onset (P21). These behavioral impairments occur due to PC dysfunction, a neuronal subpopulation with important roles in motor coordination and learning. In fact, PCs are vulnerable and easily damaged leading to impaired motor control ability.

In order to explore the impact of miR-ATXN3 treatment on transgenic mice behavior, both treated and control animals, i.e. which received a P1 intravenous injection of rAAV9 vectors encoding miR-ATXN3 or miR-Control, respectively, were subjected to a battery of tests at three different ages: 35, 55 and 85 days (FIG. 9A). These tests included stationary and accelerated rotarod, as well as beam walking test, since they are appropriate to assess balance and motor coordination. Additionally, the swimming test allowed further evaluation of motor performance and strength. On the other hand, footprint analysis allowed us to evaluate MJD-associated gait deficits.

Rotarod performance was determined as the mean latency time to fall when mice walk in a rotarod apparatus both at constant and accelerated velocities. The treatment proved to have beneficial effects at all time points and for both paradigms (FIGS. 9B and C). The most consistent results were obtained at 85 days, since this improvement was statistically significant for both stationary and accelerated rotarod (1.7 and 1.5-fold increase in latency time to fall, respectively).

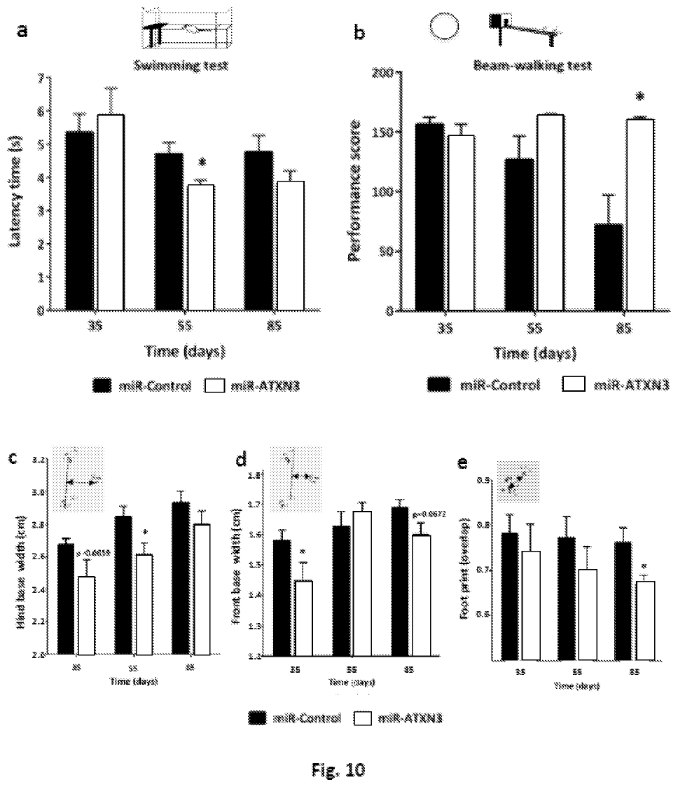

In the swimming test, mice were placed at one extremity of a water-filled glass tank and were encouraged to swim across the pool and climb a platform. The time required for each animal to swim the whole distance and climb the platform was recorded. According to the results, treated animals showed a better performance at 55 days (FIG. 10A).

In the beam-walking test, mice crossed a i) 18-mm square wide, ii) 9-mm square wide and a iii) 9-mm diameter round elevated beam. Animals were evaluated based on the time they took to complete the walk and on their motor coordination. Performance was scored according to a predefined rating scale, in which higher scores indicate a better balance and coordination. According to this analysis, no differences between the control and treated groups were detected for the 18-mm and 9-mm square wide beams. Nevertheless, animals exhibited distinct performances on the 9-mm diameter round beam, which is considered the most difficult to cross (FIG. 10B). In the control group, a progressive reduction in the performance score along time was observed, while treated mice retained their ability to traverse the beam. As a result, animals injected with rAAV9-miR-ATXN3 presented a significantly better performance in the beam-walking test at the last time point (2.2-fold increase in mean score) (FIG. 10B).

In order to assess whether the treatment was able to attenuate MJD characteristic limb and gait ataxia, the footprint pattern of both experimental groups was analyzed. Ataxic gait is normally characterized by: i) an increased stride width; ii) a shorter stride length and iii) an increased overlap distance, which reflects reduced uniformity of step alternation. Analysis of gait patterns from treated animals, when compared to the control group revealed several improvements at different time points, mainly: a significant decrease in hind and front base width, at 55 and 35 days, respectively. Additionally, at the last time point (85 days), a significant reduction in footprint overlap distance was detected (FIGS. 10C, D and E).

Overall, treated animals showed a better performance in all behavioral tests, with significant results in rotarod, swimming, beam walking and footprint analysis, indicating a general improvement in motor skills (FIGS. 9 and 10). This is the first report of significant behavioral improvement following AAV-mediated ataxin-3 silencing and the first time that rAAV9-IV injection demonstrated a positive behavioral impact in PolyQ disorders.

Altogether, this indicates a superior therapeutic impact for our strategy, possibly due to the selectivity of the SNP-targeting dsRNAs of the present disclosure, selected serotype, and delivery route

Subsequently, the impact of rAAV9-miR-ATXN3 injection on MJD-associated neuropathological changes was also evaluated. One of the major hallmarks of MJD consists on the accumulation of mutant ataxin-3 aggregates, which reflects disease progression. In the selected mouse model, these aggregates are formed in PCs starting at P40 and markedly increasing in number and size along time.

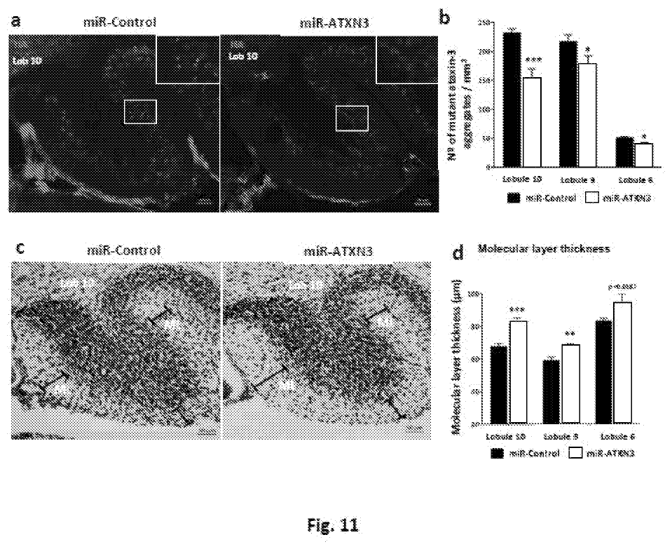

Therefore, an immunofluorescence for haemagglutinin (HA) in sagittal sections from treated and control MJD mice was performed, since this tag is present in the N-terminal of mutant ataxin-3 (FIG. 11A). Then, the number of mutant ataxin-3 aggregates per area in cerebellar lobules 10 and 9 were quantified, since they correspond to the region with higher transduction levels. In order to evaluate the impact of rAAV9 treatment in regions with low transduction efficiency, lobule 6 was also analyzed. According to the results now disclosed, miR-ATXN3 treatment reduced aggregation in all three analyzed lobules (35%, 18% and 20% decrease in lobules 10, 9 and 6, respectively), thereby contributing to neuropathology attenuation (FIG. 11B).

Another important feature in MJD patients includes cerebellar atrophy, which occurs as a consequence of neurodegeneration in this region and normally presents a correlation with clinical symptoms. In this particular mouse model, a marked cerebellar atrophy is detected as early as 3 weeks of age. Accordingly, degeneration or functional/morphological alternations in PCs might affect other cerebellar regions due to the strong interconnection between distinct cellular types. In particular, Q69 transgenic mice are characterized by a poor dendritic arborization in PCs, consequently reducing the molecular layer thickness. Therefore, cresyl violet staining was performed in sagittal sections from both experimental groups, in order to distinguish the cerebellar layers (FIG. 11C). By analyzing the molecular layer, a significant larger thickness was found in lobules 10 and 9 of miR-ATXN3 treated mice (21% and 15% respectively), as well as a strong tendency in lobule 6 (13%, p=0.0587) (FIG. 11D).

Altogether, the results now disclosed demonstrate that mutant ataxin-3 silencing through rAAV9 IV injection is an efficient therapeutic approach in transgenic MJD mice, alleviating both behavioral and neuropathological impairments. Importantly, these positive effects were obtained in a very severe model with an early onset, which could already exhibit neurological and vascularization defects on the day of birth. Therefore, an even more significant impact can be predicted if testing this strategy in other MJD models, which present a late and mild phenotype.

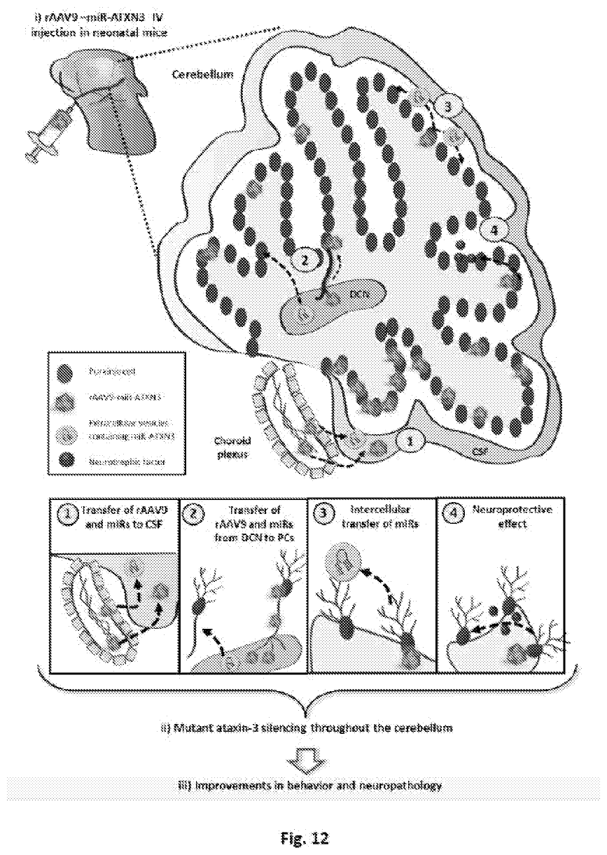

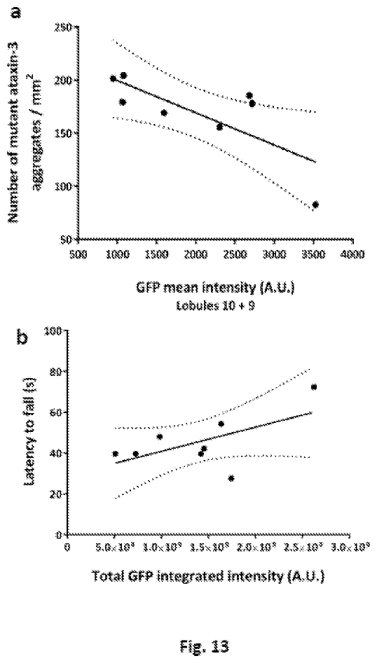

Although the first observations regarding rAAV9 distribution in MJD transgenic mice suggested a localized therapeutic response only in lobule 10, a very generalized effect was detected in the whole cerebellum and mice behavior. One could speculate that lobule 10 highly efficient transduction could be sufficient to induce improvements in beam walking test or rotarod, since this lobule is part of the vestibular system, being important for balance. However, only an overall beneficial effect could explain the general better performance of treated mice, especially in tests exploring motor coordination, strength and gait. One possible explanation is that transduced PCs in other lobules, although scarce, might be sufficient to induce positive effects in the respective region. This can occur through a neuroprotective action induced by rAAV9-positive PCs in the entire cerebellum, by releasing neurotrophic factors or inhibiting neuroinflammation, for example. Alternatively, transduced PCs might transfer miR-ATXN3 molecules to the neighboring cells. Therefore, transduced PCs might communicate with rAAV9-negative neurons through the possible transfer of solo miRs and/or using extracellular vesicles containing miR-ATXN3. Using a similar mechanism, transduced cells in the DCN can also release miR-ATXN3 constructs, which are then delivered to PC projections. Finally, the fact that CP epithelial cells are themselves transduced by rAAV9 could contribute to our findings. Accordingly, CP-directed gene therapy has been investigated in the context of lysosomal storage disorders, where it allows the continuous secretion of therapeutic proteins into CSF, leading to beneficial effects. Similarly, CP epithelial cells can secrete miRNAs incorporated into extracellular vesicles. Based on that, it is possible that rAAV9-positive CP cells in the fourth ventricle can transfer miR-ATXN3-containing extracellular vesicles to the CSF, which then exert their silencing action in the cerebellum. FIG. 12 summarizes the possible mechanisms underlying rAAV9-miR-ATXN3 therapeutic impact in the present disclosure.

It was also assessed whether the therapeutic effect in treated mice is dependent on the levels of rAAV9 cerebellar transduction. The fact that particular animals presented a more evident behavioral improvement or neuropathology attenuation could be explained by a higher vector dose transducing PCs.

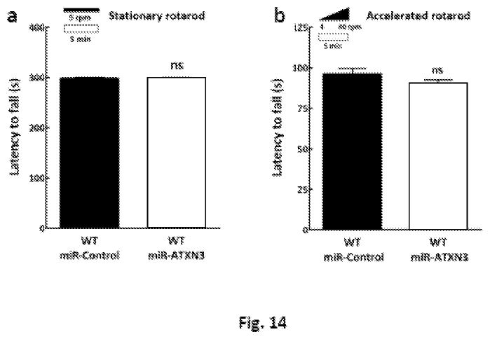

For that purpose, GFP mean fluorescence was analyzed on lobules 10 and 9, as well as the number of aggregates per area in the respective region. An inverse correlation between these two parameters was found, leading to the conclusion that higher transduction levels on lobules 10 and 9 are accompanied by an improvement in aggregate clearance (FIG. 13A). However, the same relation could not be established for lobule 6, indicating that beneficial effects on this particular region might depend on other parameters.

Moreover, it was assessed whether mice with superior cerebellar transduction correspond to the ones with better motor performance. In this context, a positive relation between GFP integrated intensity in all cerebellar lobules and average performance in accelerated rotarod was found (FIG. 13B).

Taking all of this into account, it was concluded that the variability observed in treated animals for behavioral tests and neuropathological signs can be caused by different transduction efficiencies. This can be due to the technical demand of intrafacial administration in neonatal mice, combined with the large injected volume, or the fact that some animals could have received different vector doses. Moreover, the quantity of viral particles that reach the cerebellum may also vary between different animals, possibly due to differences in vascularization or AAV receptor levels.

In summary, rAAV9-miR-ATXN3 injection induces a dose-dependent response, since higher vector concentrations in the cerebellum correspond to a more powerful therapeutic effect. So, based on these results it can be concluded that the therapeutic effect could potentially be maximized by increasing injected vector doses, i.e. the number of viral particles per animal.

Apart from the proved efficacy, the safety profile of a therapeutic strategy needs to be assessed to enable a possible translation to the clinic. Recent studies have reported immune responses triggered in the brain after rAAV9 delivery and toxicity due to miR-induced off-target silencing. Although we have not explored all these parameters in detail, the therapeutic strategy now disclosed was evaluated in wild-type animals, based on their stationary and accelerated rotarod performances at 85 days, to assess whether this treatment is well-tolerated. No differences were detected for the rotarod performance of wild-type mice intravenously injected with rAAV9-miR-Control or rAAV9-miR-ATXN3 (FIGS. 14 A and B). These findings indicate that the therapeutic sequence does not induce major toxic effects.

Finally, at PN75, animals also underwent Magnetic Resonance Imaging (MRI) and Spectroscopy (MRS) to evaluate morphological and metabolic changes of cerebellum of both treated and control MJD transgenic mice, as well as wild-type littermates using a 9.4 Tesla scanner.

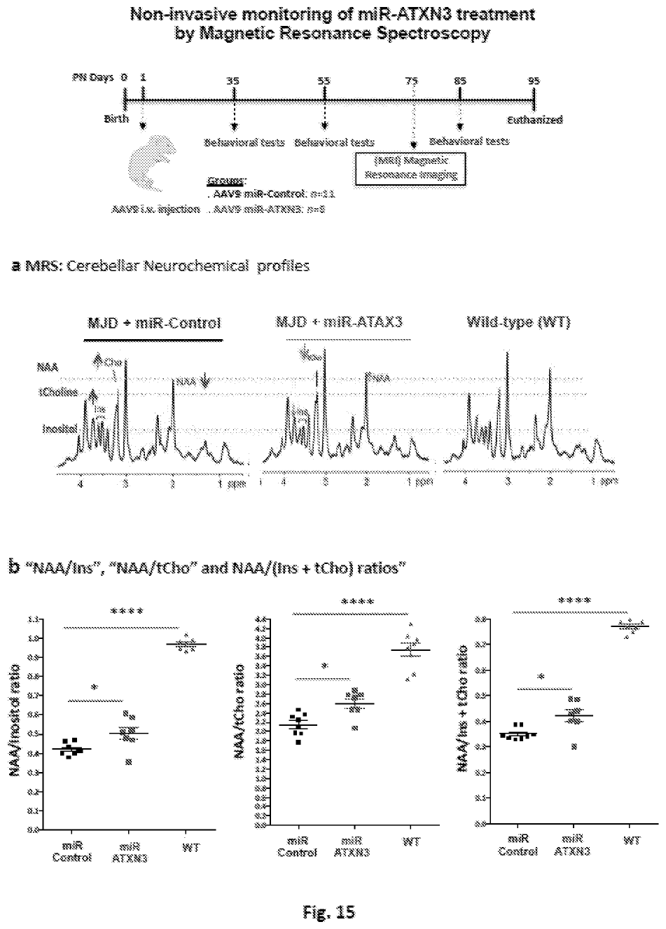

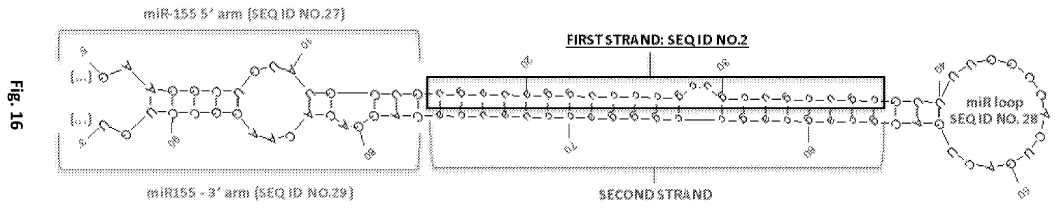

Three neurochemicals were highly deregulated in transgenic MJD when compared to wild-type mice in the cerebellum: i.e. N-acetylaspartate (NAA), myo-inositol (Ins) and glycerophosphocholine phosphocholine (tCho) (FIG. 15A). Interestingly, the levels of these three neurometabolites were ameliorated in MJD mice injected with miR-ATAX3, which means higher levels of NAA (neuronal marker) and lower levels of Ins and tCho (markers of cell death) when compared to control D mice (FIG. 15B).

Neurochemical ratios NAA/Ins and NAA/total Choline; as well as NAA/(Ins+tCho) ratio, were also applied to evaluate the efficacy of this therapy. All three ratios values were significantly higher in treated MJD mice when compared to control MJD mice, demonstrating the efficacy of miR-ATAX3 treatment (FIG. 15B).

Altogether this demonstrates that neurochemical biomarkers, in particular NAA, Ins and tCho, can be used to monitor the efficacy of this gene-based therapy during preclinical trials and subsequently be translated to human clinical trials, as important non-invasive therapeutic biomarkers.

In conclusion, this disclosure provides compelling evidence that a single intravenous injection of rAAV9 encoding a miR155-based artificial miRNA comprising SEQ ID NO.2 at P1 is able to: i) transpose the blood-brain barrier, ii) precisely silence mutant ataxin-3 mRNA and iii) alleviate MJD neuropathological changes and motor impairments.

Furthermore, the present disclosure reports a significant behavioral improvement in polyglutamine disorders following rAAV9 intravenous administration and constitutes the first MJD therapeutic approach capable of inducing widespread and long-term ataxin-3 silencing through a non-invasive system.

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

The disclosure should not be seen in any way restricted to the embodiments described and a person with ordinary skill in the art will foresee many possibilities to modifications thereof. The above described embodiments are combinable.

The following claims further set out particular embodiments of the disclosure.

SEQUENCE LISTING:
1) Target sequences and double stranded RNAs sequences targeting ATXN3-resident SNPs
1.1 Target sequences at exon 10 (r512895357) and respective SNP-targeting double-stranded RNAs
1.1.1 Target sequence and anti-sense sequence of the double stranded RNAs targeting ataxin-3
mRNA at r512895357(Cytosine)
SEQ ID NO. 1: Target sequence at exon 10 (rs12895357)(C): agcagcagcag [symbol] ggaccuauca
SEQ ID NO. 2: (miR357C.22): 5′-ugauaggucccgcugcugcugc-3′ (22 nt)
SEQ ID NO. 3: (miR357C.21): 5′-ugauaggucccgcugcugcug-3′ (21 nt)
SEQ ID NO. 4: (miR357C.19): 5′-ugauaggucccgcugcugc-3′ (19 nt)
SEQ ID NO. 5: (miR357C.20): 5′-ugauaggucccgcugcugcu-3′ (20 nt)
SEQ ID NO. 6: (miR357C.23): 5′-ugauaggucccgcugcugcugcu-3′ (23 nt)
1.1.2. Target sequence and anti-sense sequence of the double stranded RNAs targeting ataxin-3
mRNA at rs12895357 (Guanine)
SEQ ID NO. 7: Target sequence at exon 10 (rs12895357)(G): agcagcagcag [symbol] gggaccuauca
SEQ ID NO. 8 (miR357G.22): 5′-ugauaggucccccugcugcugc-3′ (22 nt)
SEQ ID NO. 9 (miR357G.19): 5′-ugauaggucccccugcugc-3′ (19 nt)
SEQ ID NO. 10 (miR357G.20): 5′-ugauaggucccccugcugcu-3′ (20 nt)
SEQ ID NO. 11 (miR357G.21): 5′-ugauaggucccccugcugcug-3′ (21 nt)
SEQ ID NO. 12 (miR357G.23): 5′-ugauaggucccccugcugcugcu-3′ (23 nt)
1.2 Target sequences at exon 8 (r51048755) and respective SNP-targeting double-stranded RNAs
1.2.1 Target sequence and anti-sense sequence of the double stranded RNAs targeting ataxin-3
allele at rs1048755 (Adenine)
SEQ ID NO. 13: Target sequence at exon 8 (rs1048755) (A): accuggaacga [symbol] uguuagaagca
SEQ ID NO. 14 (miR755A.22): 5′-ugcuucuaacauucguuccagg-3′ (22 nt)
SEQ ID NO. 15 (miR755A.19): 5′-ugcuucuaacauucguucc-3′ (19 nt)
SEQ ID NO. 16 (miR755A.20): 5′-ugcuucuaacauucguucca-3′ (20 nt)
SEQ ID NO. 17 (miR755A.21): 5′-ugcuucuaacauucguuccag-3′ (21 nt)
SEQ ID NO. 18 (miR755A.23): 5′-ugcuucuaacauucguuccaggu-3′ (23 nt)
1.2.2 Target sequence and anti-sense sequence of the double stranded RNAs targeting ataxin-3
allele at rs1048755 (Guanine)
SEQ ID NO. 19: Target sequence at exon 8 (rs1048755) (G): accuggaacga [symbol] uguuagaagca
SEQ ID NO. 20 (miR755G.22): 5′-ugcuucuaaca [symbol] ucguuccagg-3′ (22 nt)
SEQ ID NO. 21 (miR755G.19): 5′-ugcuucuaaca [symbol] ucguucc-3′ (19 nt)
SEQ ID NO. 22 (miR755G.20): 5′-ugcuucuaaca [symbol] ucguucca-3′ (20 nt)
SEQ ID NO. 23 (miR755G.21): 5′-ugcuucuaaca [symbol] ucguuccag-3′ (21 nt)
SEQ ID NO. 24 (miR755G.23): 5′-ugcuucuaaca [symbol] ucguuccaggu-3′ (23 nt)
Other Sequences
SEQ ID NO. 25 (miR-Control): 5′-caacaagaugaagagcaccaa-3′ (21 nt)
SEQ ID NO. 26 (sh-ATXN3): 5′-gauaggucccgcugcugcu-3′ (19 nt)
SEQ ID NO. 27 (miR155-5′arm): 5′-cuggaggcuugcugaaggcuguaugcug-3′
SEQ ID NO. 28 (miR loop): 5′-guuuuggccacugacugac-3′ (19 nt)
SEQ ID NO. 29 (miR155-3′ arm): 5′-caggacaaggccuguuacuagcacucacauggaacaaauggcc-3′ (43 nt)

### REFERENCES

U.S. Ser. No. 10/072,264B2
WO2005105995
Alves, S., Nascimento-Ferreira, I., Auregan, G., Hassig, R., Dufour, N., Brouillet, E., Pedroso de Lima, M. C., Hantraye, P., Pereira de Almeida, L., and Deglon, N. (2008a). Allele-specific RNA silencing of mutant ataxin-3 mediates neuroprotection in a rat model of Machado-Joseph disease. PloS one 3, e3341
Alves, S., Nascimento-Ferreira, I., Dufour, N., Hassig, R., Auregan, G., Nobrega, C., Brouillet, E., Hantraye, P., Pedroso de Lima, M. C., Deglon, N., et al. (2010). Silencing ataxin-3 mitigates degeneration in a rat model of Machado-Joseph disease: no role for wild-type ataxin-3? Human molecular genetics 19, 2380-2394
Nobrega, C., Nascimento-Ferreira, I., Onofre, I., Albuquerque, D., Hirai, H., Deglon, N., and de Almeida, L. P. (2013). Silencing mutant ataxin-3 rescues motor deficits and neuropathology in Machado-Joseph disease transgenic mice. PloS one 8, e52396
Alves, S., Regulier, E., Nascimento-Ferreira, I., Hassig, R., Dufour, N., Koeppen, A., Carvalho, A. L., Simoes, S., de Lima, M. C., Brouillet, E., et al. (2008b). Striatal and nigral pathology in a lentiviral rat model of Machado-Joseph disease. Human molecular genetics 17, 2071-2083
K. H. Chung, C. C. Hart, S. Al-Bassam et al., “Polycistronic RNA polymerase II expression vectors for RNA interference based on BIC/miR-155,” Nucleic Acids Research, vol. 34, no. 7, article e53, 2006

1. Double stranded RNA comprising a first strand of RNA and a second strand of RNA, wherein:
the first strand of RNA and the second strand of RNA are substantially complementary to each other, preferably the first and the second strand of RNA are at least 90% complementary to each other;
the first strand of RNA has a sequence length of at least 19 nucleotides;
the first strand of RNA is at least 86% complementary to SEQ ID NO. 1, 7, 13 or 19;
the first strand of RNA is different from SEQ ID NO. 26; and
a first nucleotide of the first strand of RNA is different from cytosine.

2. Double stranded RNA according to the previous claim, wherein the first strand of RNA has a sequence length of at least 19 nucleotides to 23 nucleotides.

3. Double stranded RNA according to any of the previous claims, wherein the first strand of RNA has a sequence length of 20-22 nucleotides, preferably has a sequence length of 21-22 nucleotides, more preferably has a sequence length of 22 nucleotides.

4. Double stranded RNA according to any of the previous claims, wherein the first strand of RNA is 90% identical to SEQ ID NO. 2, 3, 4, 5, 6, 8, 9, 10, 11, 12, 14, 15, 16, 17, 18, 20, 21, 22, 23 or 24; preferably 95% identical to SEQ ID NO. 2, 3, 4, 5, 6, 8, 9, 10, 11, 12, 14, 15, 16, 17, 18, 20, 21, 22, 23 or 24; more preferably 99% identical to SEQ ID NO. 2, 3, 4, 5, 6, 8, 9, 10, 11, 12, 14, 15, 16, 17, 18, 20, 21, 22, 23 or 24.

5. Double stranded RNA according to the previous claim, wherein the first strand of RNA is at least 90% complementary to SEQ ID NO. 1, 7, 13 or 19, preferably 95% complementary to SEQ ID NO. 1, 7, 13 or 19, more preferably 100% complementary to SEQ ID NO. 1, 7, 13 or 19, even more preferably the first strand of RNA is complementary to SEQ ID NO. 1, 7, 13 or 19.

6. Double stranded RNA according to any of the previous claims, wherein the first strand of RNA is selected from SEQ ID NO. 2, 3, 4, 5, 6, 8, 9, 10, 11, 12, 14, 15, 16, 17, 18, 20, 21, 22, 23 or 24.

7. Double stranded RNA according to any of the previous claims, wherein the first strand of RNA is complementary to SEQ ID NO. 1 and wherein the first strand of RNA is selected from SEQ ID NO. 2, 3, 4, 5 or 6.

8. Double stranded RNA according to any of the previous claims, wherein the first strand of RNA is SEQ ID NO. 2.

9. Double stranded RNA according to any of the previous claims 1-7, wherein the first strand of RNA is SEQ ID NO. 3.

10. Double stranded RNA according to any of the previous claims 1-6, wherein the first strand of RNA is complementary to SEQ ID NO. 7 and wherein the first strand of RNA is selected from SEQ ID NO. 8, 9, 10, 11 or 12.

11. Double stranded RNA according to any of the previous claims 1-6, wherein the first strand of RNA is complementary to SEQ ID NO. 13 and wherein the first strand of RNA is selected from SEQ ID NO. 14, 15, 16, 17 or 18.

12. Double stranded RNA according to any of the previous claims 1-6, wherein the first strand of RNA is complementary to SEQ ID NO. 19 and wherein the first strand of RNA is selected from SEQ ID NO. 20, 21, 22, 23 or 24.

13. Double stranded RNA according to any of the previous claims, wherein the first nucleotide of the first strand of RNA is uracil.

14. Double stranded RNA according to any of the previous claims, wherein the double stranded RNA is comprised in a pre-miRNA scaffold, a pri-miRNA scaffold, a miRNA scaffold, a shRNA or a siRNA, preferably a miRNA scaffold or a shRNA, more preferably a miRNA.

15. Double stranded RNA according to the previous claim, wherein the double stranded RNA is comprised in a miRNA scaffold derived from miR-155.

16. Isolated DNA sequence encoding a double stranded RNA according to any of the previous claims.

17. Expression cassette comprising the isolated DNA sequence according to claim 16 or the double stranded RNA according to any of the claims 1-15.

18. Vector comprising the isolated DNA according to claim 16 or the double stranded RNA according to any of the claims 1-15 or the expression cassette according to claim 17.

19. Vector according to the previous claim, wherein said vector is an adeno-associated viral vector or a lentiviral vector or an adenoviral vector or a non-viral vector.

20. Vector according to the previous claim, wherein the adeno-associated viral vector is AAV9 or AAVrh10 or PHP.B or PHP.eB or PHP.S.

21. Host cell comprising the isolated DNA sequence of claim 16 or the double stranded RNA of any of claims 1-15 or the expression cassette of claim 17 or the vector of any of the claims 18-19.

22. Host cell according to the previous claim, wherein the host cell is a eukaryotic cell, preferably a mammalian cell.

23. Composition comprising the isolated DNA sequence of claim 16 or the double stranded RNA of any of claims 1-15 or the expression cassette of claim 17 or the vector of any of the claims 18-19 or the host cell of any of the claims 21-22.

24. Kit comprising the isolated DNA sequence of claim 16 or the double stranded RNA of any of claims 1-15 or the expression cassette of claim 17 or the vector of any of the claims 18-19 or the host cell of any of the claims 21-22 or the composition of claim 23.

25. Double stranded RNA according to any of the claims 1-15, or a vector comprising an isolated DNA according to claim 16 or an expression cassette according to claim 17 for use in medicine.

26. Double stranded RNA according to any of the claims 1-15 or a vector comprising an isolated DNA according to claim 16 or an expression cassette according to claim 17 for use in the treatment or in the prevention of a neurodegenerative disease, or for use in the treatment or in the prevention of cytotoxic effects of said neurodegenerative disease.

27. Double stranded RNA or vector or expression cassette, for use according to the previous claim, wherein the double stranded RNA or the vector or the expression cassette is administrated to regulate the levels of neurometabolites, preferably to increase N-acetylaspartate, to decrease myo-inositol, glycerophosphocholine and phosphocholine.

28. Double stranded RNA or vector or expression cassette, for use according to any of the previous claims 26-27, wherein the neurodegenerative disease is a trinucleotide-repeat disease, preferably a CAG trinucleotide-repeat disease.

29. Double stranded RNA or vector or expression cassette, for use according to the previous claim, wherein the neurodegenerative disease is the Machado-Joseph disease.

30. Double stranded RNA or vector or expression cassette, for use according to any of the previous claims 25-28, wherein the double stranded RNA is administrated systemically, intravenously, intratumorally, orally, intranasally, intraperitoneally, intramuscularly, intravertebrally, intracerebrally, intracerebroventriculally, intracisternally, intrathecally, intraocularly, intracardiacally, intradermally, or subcutaneously, preferably intravenously, intracisternally, intrathecally or, in situ, by intracerebral administration.
